(19) United States
(12) Patent Application Publication
(10) Pub. No.: US 2003/0166706 A1
(43) Pub. Date: Sep. 4, 2003

(54) Method of treating osteoarthritis

(76) Inventors: Kenneth S. Kilgore, Ann Arbor, MI (US); Denis J. Schrier, Ann Arbor, MI (US)

Correspondence Address: WARNER-LAMBERT COMPANY, 2800 PLYMOUTH RD, ANN ARBOR, MI, 48105, US

(21) Appl. No.: 10/371,244

(22) Filed: Feb. 20, 2003

Related U.S. Application Data
(60) Provisional application No. 60/360,756, filed on Mar. 1, 2002.

(51) Int. Cl. A61K 31/405

(52) U.S. Cl. 514/419

(57) ABSTRACT

This invention relates to a method of preventing and treating osteoarthritis and inhibiting cartilage damage by administering a compound which is a 2,3,3a,4,5,6,7,7a-octahydroindol-2-carboxylic acid, or a pharmaceutically acceptable salt thereof, and a pharmaceutical composition comprising the compound or salt thereof.

### CROSS-REFERENCE TO RELATED APPLICATIONS

This application claims benefit of priority from U.S. Provisional patent application Ser. No. 60/360,756, filed Mar. 1, 2002, which is hereby incorporated herein by reference.

### BACKGROUND OF THE INVENTION

This invention relates to a method of preventing and treating osteoarthritis (“OA”) and inhibiting cartilage damage by administering a compound which is a 2,3,3a,4,5,6,7,7a-octahydroindol-2-carboxylic acid, or a pharmaceutically acceptable salt thereof, and a pharmaceutical composition comprising the compound or salt thereof.

Many people engaged in athletic activities suffer from sprains and torn cartilage resulting from the physical activity. Further, more than 23 million Americans have some form of arthritis. Among the various forms of arthritis, osteoarthritis (“OA”) is the most prevalent, affecting 21 million Americans. Osteoarthritis is primarily a disorder of cartilage and subchondral bone, although other tissues in and around affected joints are involved. OA is a result of a complex system of interrelated mechanical, biochemical, and molecular mechanisms. No matter what the source, a patient suffering from cartilage damage experiences pain and joint stiffness leading to joint deformities, diminishment or loss of joint function, and secondarily inflammation.

Aspirin and conventional nonsteroidal anti-inflammatory drugs (NSAIDs) such as ibuprofen, diclofenac, and naproxen are the primary agents used to treat pain resulting from cartilage damage, including OA-related pain. These agents inhibit prostaglandin release by blocking cyclooxygenase-mediated conversion of cell membrane lipids from arachidonic acid. However, the therapeutic use of conventional NSAIDs is limited due to drug associated side effects, including life threatening ulceration and renal toxicity. Further, each of these drugs only treat secondary conditions associated with cartilage damage or osteoarthritis such as pain, but do not prevent or treat the primary condition, which is damage to the cartilage. Not surprisingly then, patients experiencing severe cartilage damage frequently require surgery, including joint replacement surgery.

Because traditional agents used to prevent or treat disorders and diseases with a component of cartilage damage such as osteoarthritis have major shortcomings, the need for new therapies for these diseases continues. We have now discovered that a compound which is a 2,3,3a,4,5,6,7,7a-octahydroindol-2-carboxylic acid, or a pharmaceutically acceptable salt thereof is useful for preventing and inhibiting cartilage damage, alleviating pain, and preventing and treating osteoarthritis. All that is required to prevent and/or inhibit the cartilage damage, alleviate pain, and prevent and/or treat osteoarthritis according to the invention is to administer to a subject in need of treatment an effective amount of a 2,3,3a,4,5,6,7,7a-octahydroindol-2-carboxylic acid, or a pharmaceutically acceptable salt thereof.

### SUMMARY OF THE INVENTION

This invention provides:

1. A method of inhibiting cartilage damage in a mammal, comprising administering to the mammal a cartilage damage inhibiting effective amount of a 2,3,3a,4,5,6,7,7a-octahydroindol-2-carboxylic acid, or a pharmaceutically acceptable salt thereof.

Additional embodiments of the invention method include:

2. The method according to Embodiment 1, wherein the 2,3,3a,4,5,6,7,7a-octahydroindol-2-carboxylic acid, or a pharmaceutically acceptable salt thereof, is a mixture of all eight possible stereoisomers, or a pharmaceutically acceptable salt thereof.

3. The method according to Embodiment 1, wherein the 2,3,3a,4,5,6,7,7a-octahydroindol-2-carboxylic acid, or a pharmaceutically acceptable salt thereof, is a mixture of any two of the eight possible stereoisomers, or a pharmaceutically acceptable salt thereof.

4. The method according to Embodiment 1, wherein the 2,3,3a,4,5,6,7,7a-octahydroindol-2-carboxylic acid, or a pharmaceutically acceptable salt thereof, is a mixture of any three of the eight possible stereoisomers, or a pharmaceutically acceptable salt thereof.

5. The method according to Embodiment 1, wherein the 2,3,3a,4,5,6,7,7a-octahydroindol-2-carboxylic acid, or a pharmaceutically acceptable salt thereof, is a mixture of any four of the eight possible stereoisomers, or a pharmaceutically acceptable salt thereof.

6. The method according to Embodiment 1, wherein the 2,3,3a,4,5,6,7,7a-octahydroindol-2-carboxylic acid, or a pharmaceutically acceptable salt thereof, is a mixture of any five of the eight possible stereoisomers, or a pharmaceutically acceptable salt thereof.

7. The method according to Embodiment 1, wherein the 2,3,3a,4,5,6,7,7a-octahydroindol-2-carboxylic acid, or a pharmaceutically acceptable salt thereof, is a mixture of any six of the eight possible stereoisomers, or a pharmaceutically acceptable salt thereof.

8. The method according to Embodiment 1, wherein the 2,3,3a,4,5,6,7,7a-octahydroindol-2-carboxylic acid, or a pharmaceutically acceptable salt thereof, is a mixture of any seven of the eight possible stereoisomers, or a pharmaceutically acceptable salt thereof.

9. The method according to Embodiment 1, wherein the 2,3,3a,4,5,6,7,7a-octahydroindol-2-carboxylic acid, or a pharmaceutically acceptable salt thereof, is a mixture of

[2(S), 3a(S), 7a(S)]-2,3,3a,4,5,6,7,7a-octahydroindol-2-carboxylic acid and

[2(R), 3a(R), 7a(R)]-2,3,3a,4,5,6,7,7a-octahydroindol-2-carboxylic acid, or a pharmaceutically acceptable salt thereof.

10. The method according to Embodiment 1, wherein the 2,3,3a,4,5,6,7,7a-octahydroindol-2-carboxylic acid, or a pharmaceutically acceptable salt thereof, is a mixture of

[2(R), 3a(S), 7a(S)]-2,3,3a,4,5,6,7,7a-octahydroindol-2-carboxylic acid and

[2(S), 3a(R), 7a(R)]-2,3,3a,4,5,6,7,7a-octahydroindol-2-carboxylic acid, or a pharmaceutically acceptable salt thereof.

11. The method according to Embodiment 1, wherein the 2,3,3a,4,5,6,7,7a-octahydroindol-2-carboxylic acid, or a pharmaceutically acceptable salt thereof, is a mixture of

[2(S), 3a(R), 7a(S)]-2,3,3a,4,5,6,7,7a-octahydroindol-2-carboxylic acid and

[2(R), 3a(S), 7a(R)]-2,3,3a,4,5,6,7,7a-octahydroindol-2-carboxylic acid, or a pharmaceutically acceptable salt thereof.

12. The method according to Embodiment 1, wherein the 2,3,3a,4,5,6,7,7a-octahydroindol-2-carboxylic acid, or a pharmaceutically acceptable salt thereof, is a mixture of

[2(S), 3a(S), 7a(R)]-2,3,3a,4,5,6,7,7a-octahydroindol-2-carboxylic acid and

[2(R), 3a(R), 7a(S)]-2,3,3a,4,5,6,7,7a-octahydroindol-2-carboxylic acid, or a pharmaceutically acceptable salt thereof.

13. The method according to Embodiment 1, wherein the 2,3,3a,4,5,6,7,7a-octahydroindol-2-carboxylic acid, or a pharmaceutically acceptable salt thereof, is a compound named [2(S), 3a(S), 7a(S)]-2,3,3a,4,5,6,7,7a-octahydroindol-2-carboxylic acid, or a pharmaceutically acceptable salt thereof.

14. The method according to Embodiment 1, wherein the 2,3,3a,4,5,6,7,7a-octahydroindol-2-carboxylic acid, or a pharmaceutically acceptable salt thereof, is a compound named [2(R), 3a(R), 7a(R)]-2,3,3a,4,5,6,7,7a-octahydroindol-2-carboxylic acid, or a pharmaceutically acceptable salt thereof.

15. The method according to Embodiment 1, wherein the 2,3,3a,4,5,6,7,7a-octahydroindol-2-carboxylic acid, or a pharmaceutically acceptable salt thereof, is a compound named [2(R), 3a(S), 7a(S)]-2,3,3a,4,5,6,7,7a-octahydroindol-2-carboxylic acid, or a pharmaceutically acceptable salt thereof.

16. The method according to Embodiment 1, wherein the 2,3,3a,4,5,6,7,7a-octahydroindol-2-carboxylic acid, or a pharmaceutically acceptable salt thereof, is a compound named [2(S), 3a(R), 7a(R)]-2,3,3a,4,5,6,7,7a-octahydroindol-2-carboxylic acid, or a pharmaceutically acceptable salt thereof.

17. The method according to Embodiment 1, wherein the 2,3,3a,4,5,6,7,7a-octahydroindol-2-carboxylic acid, or a pharmaceutically acceptable salt thereof, is a compound named [2(S), 3a(R), 7a(S)]-2,3,3a,4,5,6,7,7a-octahydroindol-2-carboxylic acid, or a pharmaceutically acceptable salt thereof.

18. The method according to Embodiment 1, wherein the 2,3,3a,4,5,6,7,7a-octahydroindol-2-carboxylic acid, or a pharmaceutically acceptable salt thereof, is a compound named [2(R), 3a(S), 7a(R)]-2,3,3a,4,5,6,7,7a-octahydroindol-2-carboxylic acid, or a pharmaceutically acceptable salt thereof.

19. The method according to Embodiment 1, wherein the 2,3,3a,4,5,6,7,7a-octahydroindol-2-carboxylic acid, or a pharmaceutically acceptable salt thereof, is a compound named [2(S), 3a(S), 7a(R)]-2,3,3a,4,5,6,7,7a-octahydroindol-2-carboxylic acid, or a pharmaceutically acceptable salt thereof.

20. The method according to Embodiment 1, wherein the 2,3,3a,4,5,6,7,7a-octahydroindol-2-carboxylic acid, or a pharmaceutically acceptable salt thereof, is a compound named [2(R), 3a(R), 7a(S)]-2,3,3a,4,5,6,7,7a-octahydroindol-2-carboxylic acid, or a pharmaceutically acceptable salt thereof.

This invention also provides:

21. A method of preventing cartilage damage in a mammal, comprising administering to the mammal a cartilage damage preventing effective amount of a 2,3,3a,4,5,6,7,7a-octahydroindol-2-carboxylic acid, or a pharmaceutically acceptable salt thereof.

Additional embodiments of the invention method include:

22. The method according to Embodiment 21, wherein the 2,3,3a,4,5,6,7,7a-octahydroindol-2-carboxylic acid, or a pharmaceutically acceptable salt thereof, is a mixture of all eight possible stereoisomers, or a pharmaceutically acceptable salt thereof.

23. The method according to Embodiment 21, wherein the 2,3,3a,4,5,6,7,7a-octahydroindol-2-carboxylic acid, or a pharmaceutically acceptable salt thereof, is a mixture of any two of the eight possible stereoisomers, or a pharmaceutically acceptable salt thereof.

24. The method according to Embodiment 21, wherein the 2,3,3a,4,5,6,7,7a-octahydroindol-2-carboxylic acid, or a pharmaceutically acceptable salt thereof, is a mixture of any three of the eight possible stereoisomers, or a pharmaceutically acceptable salt thereof.

25. The method according to Embodiment 21, wherein the 2,3,3a,4,5,6,7,7a-octahydroindol-2-carboxylic acid, or a pharmaceutically acceptable salt thereof, is a mixture of any four of the eight possible stereoisomers, or a pharmaceutically acceptable salt thereof.

26. The method according to Embodiment 21, wherein the 2,3,3a,4,5,6,7,7a-octahydroindol-2-carboxylic acid, or a pharmaceutically acceptable salt thereof, is a mixture of any five of the eight possible stereoisomers, or a pharmaceutically acceptable salt thereof.

27. The method according to Embodiment 21, wherein the 2,3,3a,4,5,6,7,7a-octahydroindol-2-carboxylic acid, or a pharmaceutically acceptable salt thereof, is a mixture of any six of the eight possible stereoisomers, or a pharmaceutically acceptable salt thereof.

28. The method according to Embodiment 21, wherein the 2,3,3a,4,5,6,7,7a-octahydroindol-2-carboxylic acid, or a pharmaceutically acceptable salt thereof, is a mixture of any seven of the eight possible stereoisomers, or a pharmaceutically acceptable salt thereof.

29. The method according to Embodiment 21, wherein the 2,3,3a,4,5,6,7,7a-octahydroindol-2-carboxylic acid, or a pharmaceutically acceptable salt thereof, is a mixture of

[2(S), 3a(S), 7a(S)]-2,3,3a,4,5,6,7,7a-octahydroindol-2-carboxylic acid and

[2(R), 3a(R), 7a(R)]-2,3,3a,4,5,6,7,7a-octahydroindol-2-carboxylic acid, or a pharmaceutically acceptable salt thereof.

30. The method according to Embodiment 21, wherein the 2,3,3a,4,5,6,7,7a-octahydroindol-2-carboxylic acid, or a pharmaceutically acceptable salt thereof, is a mixture of

[2(R), 3a(S), 7a(S)]-2,3,3a,4,5,6,7,7a-octahydroindol-2-carboxylic acid and

[2(S), 3a(R), 7a(R)]-2,3,3a,4,5,6,7,7a-octahydroindol-2-carboxylic acid, or a pharmaceutically acceptable salt thereof.

31. The method according to Embodiment 21, wherein the 2,3,3a,4,5,6,7,7a-octahydroindol-2-carboxylic acid, or a pharmaceutically acceptable salt thereof, is a mixture of

[2(S), 3a(R), 7a(S)]-2,3,3a,4,5,6,7,7a-octahydroindol-2-carboxylic acid and

[2(R), 3a(S), 7a(R)]-2,3,3a,4,5,6,7,7a-octahydroindol-2-carboxylic acid, or a pharmaceutically acceptable salt thereof.

32. The method according to Embodiment 21, wherein the 2,3,3a,4,5,6,7,7a-octahydroindol-2-carboxylic acid, or a pharmaceutically acceptable salt thereof, is a mixture of

[2(S), 3a(S), 7a(R)]-2,3,3a,4,5,6,7,7a-octahydroindol-2-carboxylic acid and

[2(R), 3a(R), 7a(S)]-2,3,3a,4,5,6,7,7a-octahydroindol-2-carboxylic acid, or a pharmaceutically acceptable salt thereof.

33. The method according to Embodiment 21, wherein the 2,3,3a,4,5,6,7,7a-octahydroindol-2-carboxylic acid, or a pharmaceutically acceptable salt thereof, is a compound named [2(S), 3a(S), 7a(S)]-2,3,3a,4,5,6,7,7a-octahydroindol-2-carboxylic acid, or a pharmaceutically acceptable salt thereof.

34. The method according to Embodiment 21, wherein the 2,3,3a,4,5,6,7,7a-octahydroindol-2-carboxylic acid, or a pharmaceutically acceptable salt thereof, is a compound named [2(R), 3a(R), 7a(R)]-2,3,3a,4,5,6,7,7a-octahydroindol-2-carboxylic acid, or a pharmaceutically acceptable salt thereof.

35. The method according to Embodiment 21, wherein the 2,3,3a,4,5,6,7,7a-octahydroindol-2-carboxylic acid, or a pharmaceutically acceptable salt thereof, is a compound named [2(R), 3a(S), 7a(S)]-2,3,3a,4,5,6,7,7a-octahydroindol-2-carboxylic acid, or a pharmaceutically acceptable salt thereof.

36. The method according to Embodiment 21, wherein the 2,3,3a,4,5,6,7,7a-octahydroindol-2-carboxylic acid, or a pharmaceutically acceptable salt thereof, is a compound named [2(S), 3a(R), 7a(R)]-2,3,3a,4,5,6,7,7a-octahydroindol-2-carboxylic acid, or a pharmaceutically acceptable salt thereof.

37. The method according to Embodiment 21, wherein the 2,3,3a,4,5,6,7,7a-octahydroindol-2-carboxylic acid, or a pharmaceutically acceptable salt thereof, is a compound named [2(S), 3a(R), 7a(S)]-2,3,3a,4,5,6,7,7a-octahydroindol-2-carboxylic acid, or a pharmaceutically acceptable salt thereof.

38. The method according to Embodiment 21, wherein the 2,3,3a,4,5,6,7,7a-octahydroindol-2-carboxylic acid, or a pharmaceutically acceptable salt thereof, is a compound named [2(R), 3a(S), 7a(R)]-2,3,3a,4,5,6,7,7a-octahydroindol-2-carboxylic acid, or a pharmaceutically acceptable salt thereof.

39. The method according to Embodiment 21, wherein the 2,3,3a,4,5,6,7,7a-octahydroindol-2-carboxylic acid, or a pharmaceutically acceptable salt thereof, is a compound named [2(S), 3a(S), 7a(R)]-2,3,3a,4,5,6,7,7a-octahydroindol-2-carboxylic acid, or a pharmaceutically acceptable salt thereof.

40. The method according to Embodiment 21, wherein the 2,3,3a,4,5,6,7,7a-octahydroindol-2-carboxylic acid, or a pharmaceutically acceptable salt thereof, is a compound named [2(R), 3a(R), 7a(S)]-2,3,3a,4,5,6,7,7a-octahydroindol-2-carboxylic acid, or a pharmaceutically acceptable salt thereof.

This invention also provides:

41. A method of treating osteoarthritis in a mammal, comprising administering to the mammal a therapeutically effective amount of a 2,3,3a,4,5,6,7,7a-octahydroindol-2-carboxylic acid, or a pharmaceutically acceptable salt thereof.

Additional embodiments of the invention method include:

42. The method according to Embodiment 41, wherein the 2,3,3a,4,5,6,7,7a-octahydroindol-2-carboxylic acid, or a pharmaceutically acceptable salt thereof, is a mixture of all eight possible stereoisomers, or a pharmaceutically acceptable salt thereof.

43. The method according to Embodiment 41, wherein the 2,3,3a,4,5,6,7,7a-octahydroindol-2-carboxylic acid, or a pharmaceutically acceptable salt thereof, is a mixture of any two of the eight possible stereoisomers, or a pharmaceutically acceptable salt thereof.

44. The method according to Embodiment 41, wherein the 2,3,3a,4,5,6,7,7a-octahydroindol-2-carboxylic acid, or a pharmaceutically acceptable salt thereof, is a mixture of any three of the eight possible stereoisomers, or a pharmaceutically acceptable salt thereof.

45. The method according to Embodiment 41, wherein the 2,3,3a,4,5,6,7,7a-octahydroindol-2-carboxylic acid, or a pharmaceutically acceptable salt thereof, is a mixture of any four of the eight possible stereoisomers, or a pharmaceutically acceptable salt thereof.

46. The method according to Embodiment 41, wherein the 2,3,3a,4,5,6,7,7a-octahydroindol-2-carboxylic acid, or a pharmaceutically acceptable salt thereof, is a mixture of any five of the eight possible stereoisomers, or a pharmaceutically acceptable salt thereof.

47. The method according to Embodiment 41, wherein the 2,3,3a,4,5,6,7,7a-octahydroindol-2-carboxylic acid, or a pharmaceutically acceptable salt thereof, is a mixture of any six of the eight possible stereoisomers, or a pharmaceutically acceptable salt thereof.

48. The method according to Embodiment 41, wherein the 2,3,3a,4,5,6,7,7a-octahydroindol-2-carboxylic acid, or a pharmaceutically acceptable salt thereof, is a mixture of any seven of the eight possible stereoisomers, or a pharmaceutically acceptable salt thereof.

49. The method according to Embodiment 41, wherein the 2,3,3a,4,5,6,7,7a-octahydroindol-2-carboxylic acid, or a pharmaceutically acceptable salt thereof, is a mixture of

[2(S), 3a(S), 7a(S)]-2,3,3a,4,5,6,7,7a-octahydroindol-2-carboxylic acid and

[2(R), 3a(R), 7a(R)]-2,3,3a,4,5,6,7,7a-octahydroindol-2-carboxylic acid, or a pharmaceutically acceptable salt thereof.

50. The method according to Embodiment 41, wherein the 2,3,3a,4,5,6,7,7a-octahydroindol-2-carboxylic acid, or a pharmaceutically acceptable salt thereof, is a mixture of

[2(R), 3a(S), 7a(S)]-2,3,3a,4,5,6,7,7a-octahydroindol-2-carboxylic acid and

[2(S), 3a(R), 7a(R)]-2,3,3a,4,5,6,7,7a-octahydroindol-2-carboxylic acid, or a pharmaceutically acceptable salt thereof.

51. The method according to Embodiment 41, wherein the 2,3,3a,4,5,6,7,7a-octahydroindol-2-carboxylic acid, or a pharmaceutically acceptable salt thereof, is a mixture of

[2(S), 3a(R), 7a(S)]-2,3,3a,4,5,6,7,7a-octahydroindol-2-carboxylic acid and

[2(R), 3a(S), 7a(R)]-2,3,3a,4,5,6,7,7a-octahydroindol-2-carboxylic acid, or a pharmaceutically acceptable salt thereof.

52. The method according to Embodiment 41, wherein the 2,3,3a,4,5,6,7,7a-octahydroindol-2-carboxylic acid, or a pharmaceutically acceptable salt thereof, is a mixture of

[2(S), 3a(S), 7a(R)]-2,3,3a,4,5,6,7,7a-octahydroindol-2-carboxylic acid and

[2(R), 3a(R), 7a(S)]-2,3,3a,4,5,6,7,7a-octahydroindol-2-carboxylic acid, or a pharmaceutically acceptable salt thereof.

53. The method according to Embodiment 41, wherein the 2,3,3a,4,5,6,7,7a-octahydroindol-2-carboxylic acid, or a pharmaceutically acceptable salt thereof, is a compound named [2(S), 3a(S), 7a(S)]-2,3,3a,4,5,6,7,7a-octahydroindol-2-carboxylic acid, or a pharmaceutically acceptable salt thereof.

54. The method according to Embodiment 41, wherein the 2,3,3a,4,5,6,7,7a-octahydroindol-2-carboxylic acid, or a pharmaceutically acceptable salt thereof, is a compound named [2(R), 3a(R), 7a(R)]-2,3,3a,4,5,6,7,7a-octahydroindol-2-carboxylic acid, or a pharmaceutically acceptable salt thereof.

55. The method according to Embodiment 41, wherein the 2,3,3a,4,5,6,7,7a-octahydroindol-2-carboxylic acid, or a pharmaceutically acceptable salt thereof, is a compound named [2(R), 3a(S), 7a(S)]-2,3,3a,4,5,6,7,7a-octahydroindol-2-carboxylic acid, or a pharmaceutically acceptable salt thereof.

56. The method according to Embodiment 41, wherein the 2,3,3a,4,5,6,7,7a-octahydroindol-2-carboxylic acid, or a pharmaceutically acceptable salt thereof, is a compound named [2(S), 3a(R), 7a(R)]-2,3,3a,4,5,6,7,7a-octahydroindol-2-carboxylic acid, or a pharmaceutically acceptable salt thereof.

57. The method according to Embodiment 41, wherein the 2,3,3a,4,5,6,7,7a-octahydroindol-2-carboxylic acid, or a pharmaceutically acceptable salt thereof, is a compound named [2(S), 3a(R), 7a(S)]-2,3,3a,4,5,6,7,7a-octahydroindol-2-carboxylic acid, or a pharmaceutically acceptable salt thereof.

58. The method according to Embodiment 41, wherein the 2,3,3a,4,5,6,7,7a-octahydroindol-2-carboxylic acid, or a pharmaceutically acceptable salt thereof, is a compound named [2(R), 3a(S), 7a(R)]-2,3,3a,4,5,6,7,7a-octahydroindol-2-carboxylic acid, or a pharmaceutically acceptable salt thereof.

59. The method according to Embodiment 41, wherein the 2,3,3a,4,5,6,7,7a-octahydroindol-2-carboxylic acid, or a pharmaceutically acceptable salt thereof, is a compound named [2(S), 3a(S), 7a(R)]-2,3,3a,4,5,6,7,7a-octahydroindol-2-carboxylic acid, or a pharmaceutically acceptable salt thereof.

60. The method according to Embodiment 41, wherein the 2,3,3a,4,5,6,7,7a-octahydroindol-2-carboxylic acid, or a pharmaceutically acceptable salt thereof, is a compound named [2(R), 3a(R), 7a(S)]-2,3,3a,4,5,6,7,7a-octahydroindol-2-carboxylic acid, or a pharmaceutically acceptable salt thereof.

This invention also provides:

61. A method of preventing osteoarthritis in a mammal, comprising administering to the mammal an osteoarthritis preventing effective amount of a 2,3,3a,4,5,6,7,7a-octahydroindol-2-carboxylic acid, or a pharmaceutically acceptable salt thereof.

Additional embodiments of the invention method include:

62. The method according to Embodiment 61, wherein the 2,3,3a,4,5,6,7,7a-octahydroindol-2-carboxylic acid, or a pharmaceutically acceptable salt thereof, is a mixture of all eight possible stereoisomers, or a pharmaceutically acceptable salt thereof.

63. The method according to Embodiment 61, wherein the 2,3,3a,4,5,6,7,7a-octahydroindol-2-carboxylic acid, or a pharmaceutically acceptable salt thereof, is a mixture of any two of the eight possible stereoisomers, or a pharmaceutically acceptable salt thereof.

64. The method according to Embodiment 61, wherein the 2,3,3a,4,5,6,7,7a-octahydroindol-2-carboxylic acid, or a pharmaceutically acceptable salt thereof, is a mixture of any three of the eight possible stereoisomers, or a pharmaceutically acceptable salt thereof.

65. The method according to Embodiment 61, wherein the 2,3,3a,4,5,6,7,7a-octahydroindol-2-carboxylic acid, or a pharmaceutically acceptable salt thereof, is a mixture of any four of the eight possible stereoisomers, or a pharmaceutically acceptable salt thereof.

66. The method according to Embodiment 61, wherein the 2,3,3a,4,5,6,7,7a-octahydroindol-2-carboxylic acid, or a pharmaceutically acceptable salt thereof, is a mixture of any five of the eight possible stereoisomers, or a pharmaceutically acceptable salt thereof.

67. The method according to Embodiment 61, wherein the 2,3,3a,4,5,6,7,7a-octahydroindol-2-carboxylic acid, or a pharmaceutically acceptable salt thereof, is a mixture of any six of the eight possible stereoisomers, or a pharmaceutically acceptable salt thereof.

68. The method according to Embodiment 61, wherein the 2,3,3a,4,5,6,7,7a-octahydroindol-2-carboxylic acid, or a pharmaceutically acceptable salt thereof, is a mixture of any seven of the eight possible stereoisomers, or a pharmaceutically acceptable salt thereof.

69. The method according to Embodiment 61, wherein the 2,3,3a,4,5,6,7,7a-octahydroindol-2-carboxylic acid, or a pharmaceutically acceptable salt thereof, is a mixture of

[2(S), 3a(S), 7a(S)]-2,3,3a,4,5,6,7,7a-octahydroindol-2-carboxylic acid and

[2(R), 3a(R), 7a(R)]-2,3,3a,4,5,6,7,7a-octahydroindol-2-carboxylic acid, or a pharmaceutically acceptable salt thereof.

70. The method according to Embodiment 61, wherein the 2,3,3a,4,5,6,7,7a-octahydroindol-2-carboxylic acid, or a pharmaceutically acceptable salt thereof, is a mixture of

[2(R), 3a(S), 7a(S)]-2,3,3a,4,5,6,7,7a-octahydroindol-2-carboxylic acid and

[2(S), 3a(R), 7a(R)]-2,3,3a,4,5,6,7,7a-octahydroindol-2-carboxylic acid, or a pharmaceutically acceptable salt thereof.

71. The method according to Embodiment 61, wherein the 2,3,3a,4,5,6,7,7a-octahydroindol-2-carboxylic acid, or a pharmaceutically acceptable salt thereof, is a mixture of

[2(S), 3a(R), 7a(S)]-2,3,3a,4,5,6,7,7a-octahydroindol-2-carboxylic acid and

[2(R), 3a(S), 7a(R)]-2,3,3a,4,5,6,7,7a-octahydroindol-2-carboxylic acid, or a pharmaceutically acceptable salt thereof.

72. The method according to Embodiment 61, wherein the 2,3,3a,4,5,6,7,7a-octahydroindol-2-carboxylic acid, or a pharmaceutically acceptable salt thereof, is a mixture of

[2(S), 3a(S), 7a(R)]-2,3,3a,4,5,6,7,7a-octahydroindol-2-carboxylic acid and

[2(R), 3a(R), 7a(S)]-2,3,3a,4,5,6,7,7a-octahydroindol-2-carboxylic acid, or a pharmaceutically acceptable salt thereof.

73. The method according to Embodiment 61, wherein the 2,3,3a,4,5,6,7,7a-octahydroindol-2-carboxylic acid, or a pharmaceutically acceptable salt thereof, is a compound named [2(S), 3a(S), 7a(S)]-2,3,3a,4,5,6,7,7a-octahydroindol-2-carboxylic acid, or a pharmaceutically acceptable salt thereof.

74. The method according to Embodiment 61, wherein the 2,3,3a,4,5,6,7,7a-octahydroindol-2-carboxylic acid, or a pharmaceutically acceptable salt thereof, is a compound named [2(R), 3a(R), 7a(R)]-2,3,3a,4,5,6,7,7a-octahydroindol-2-carboxylic acid, or a pharmaceutically acceptable salt thereof.

75. The method according to Embodiment 61, wherein the 2,3,3a,4,5,6,7,7a-octahydroindol-2-carboxylic acid, or a pharmaceutically acceptable salt thereof, is a compound named [2(R), 3a(S), 7a(S)]-2,3,3a,4,5,6,7,7a-octahydroindol-2-carboxylic acid, or a pharmaceutically acceptable salt thereof.

76. The method according to Embodiment 61, wherein the 2,3,3a,4,5,6,7,7a-octahydroindol-2-carboxylic acid, or a pharmaceutically acceptable salt thereof, is a compound named [2(S), 3a(R), 7a(R)]-2,3,3a,4,5,6,7,7a-octahydroindol-2-carboxylic acid, or a pharmaceutically acceptable salt thereof.

77. The method according to Embodiment 61, wherein the 2,3,3a,4,5,6,7,7a-octahydroindol-2-carboxylic acid, or a pharmaceutically acceptable salt thereof, is a compound named [2(S), 3a(R), 7a(S)]-2,3,3a,4,5,6,7,7a-octahydroindol-2-carboxylic acid, or a pharmaceutically acceptable salt thereof.

78. The method according to Embodiment 61, wherein the 2,3,3a,4,5,6,7,7a-octahydroindol-2-carboxylic acid, or a pharmaceutically acceptable salt thereof, is a compound named [2(R), 3a(S), 7a(R)]-2,3,3a,4,5,6,7,7a-octahydroindol-2-carboxylic acid, or a pharmaceutically acceptable salt thereof.

79. The method according to Embodiment 61, wherein the 2,3,3a,4,5,6,7,7a-octahydroindol-2-carboxylic acid, or a pharmaceutically acceptable salt thereof, is a compound named [2(S), 3a(S), 7a(R)]-2,3,3a,4,5,6,7,7a-octahydroindol-2-carboxylic acid, or a pharmaceutically acceptable salt thereof.

80. The method according to Embodiment 61, wherein the 2,3,3a,4,5,6,7,7a-octahydroindol-2-carboxylic acid, or a pharmaceutically acceptable salt thereof, is a compound named [2(R), 3a(R), 7a(S)]-2,3,3a,4,5,6,7,7a-octahydroindol-2-carboxylic acid, or a pharmaceutically acceptable salt thereof.

This invention also provides:

81. A method of alleviating pain in a mammal, comprising administering to the mammal a therapeutically effective amount of a 2,3,3a,4,5,6,7,7a-octahydroindol-2-carboxylic acid, or a pharmaceutically acceptable salt thereof.

Additional embodiments of the invention method include:

82. The method according to Embodiment 81, wherein the 2,3,3a,4,5,6,7,7a-octahydroindol-2-carboxylic acid, or a pharmaceutically acceptable salt thereof, is a mixture of all eight possible stereoisomers, or a pharmaceutically acceptable salt thereof.

83. The method according to Embodiment 81, wherein the 2,3,3a,4,5,6,7,7a-octahydroindol-2-carboxylic acid, or a pharmaceutically acceptable salt thereof, is a mixture of any two of the eight possible stereoisomers, or a pharmaceutically acceptable salt thereof.

84. The method according to Embodiment 81, wherein the 2,3,3a,4,5,6,7,7a-octahydroindol-2-carboxylic acid, or a pharmaceutically acceptable salt thereof, is a mixture of any three of the eight possible stereoisomers, or a pharmaceutically acceptable salt thereof.

85. The method according to Embodiment 81, wherein the 2,3,3a,4,5,6,7,7a-octahydroindol-2-carboxylic acid, or a pharmaceutically acceptable salt thereof, is a mixture of any four of the eight possible stereoisomers, or a pharmaceutically acceptable salt thereof.

86. The method according to Embodiment 81, wherein the 2,3,3a,4,5,6,7,7a-octahydroindol-2-carboxylic acid, or a pharmaceutically acceptable salt thereof, is a mixture of any five of the eight possible stereoisomers, or a pharmaceutically acceptable salt thereof.

87. The method according to Embodiment 81, wherein the 2,3,3a,4,5,6,7,7a-octahydroindol-2-carboxylic acid, or a pharmaceutically acceptable salt thereof, is a mixture of any six of the eight possible stereoisomers, or a pharmaceutically acceptable salt thereof.

88. The method according to Embodiment 81, wherein the 2,3,3a,4,5,6,7,7a-octahydroindol-2-carboxylic acid, or a pharmaceutically acceptable salt thereof, is a mixture of any seven of the eight possible stereoisomers, or a pharmaceutically acceptable salt thereof.

89. The method according to Embodiment 81, wherein the 2,3,3a,4,5,6,7,7a-octahydroindol-2-carboxylic acid, or a pharmaceutically acceptable salt thereof, is a mixture of

[2(S), 3a(S), 7a(S)]-2,3,3a,4,5,6,7,7a-octahydroindol-2-carboxylic acid and

[2(R), 3a(R), 7a(R)]-2,3,3a,4,5,6,7,7a-octahydroindol-2-carboxylic acid, or a pharmaceutically acceptable salt thereof.

90. The method according to Embodiment 81, wherein the 2,3,3a,4,5,6,7,7a-octahydroindol-2-carboxylic acid, or a pharmaceutically acceptable salt thereof, is a mixture of

[2(R), 3a(S), 7a(S)]-2,3,3a,4,5,6,7,7a-octahydroindol-2-carboxylic acid and

[2(S), 3a(R), 7a(R)]-2,3,3a,4,5,6,7,7a-octahydroindol-2-carboxylic acid, or a pharmaceutically acceptable salt thereof.

91. The method according to Embodiment 81, wherein the 2,3,3a,4,5,6,7,7a-octahydroindol-2-carboxylic acid, or a pharmaceutically acceptable salt thereof, is a mixture of

[2(S), 3a(R), 7a(S)]-2,3,3a,4,5,6,7,7a-octahydroindol-2-carboxylic acid and

[2(R), 3a(S), 7a(R)]-2,3,3a,4,5,6,7,7a-octahydroindol-2-carboxylic acid, or a pharmaceutically acceptable salt thereof.

92. The method according to Embodiment 81, wherein the 2,3,3a,4,5,6,7,7a-octahydroindol-2-carboxylic acid, or a pharmaceutically acceptable salt thereof, is a mixture of

[2(S), 3a(S), 7a(R)]-2,3,3a,4,5,6,7,7a-octahydroindol-2-carboxylic acid and

[2(R), 3a(R), 7a(S)]-2,3,3a,4,5,6,7,7a-octahydroindol-2-carboxylic acid, or a pharmaceutically acceptable salt thereof.

93. The method according to Embodiment 81, wherein the 2,3,3a,4,5,6,7,7a-octahydroindol-2-carboxylic acid, or a pharmaceutically acceptable salt thereof, is a compound named [2(S), 3a(S), 7a(S)]-2,3,3a,4,5,6,7,7a-octahydroindol-2-carboxylic acid, or a pharmaceutically acceptable salt thereof.

94. The method according to Embodiment 81, wherein the 2,3,3a,4,5,6,7,7a-octahydroindol-2-carboxylic acid, or a pharmaceutically acceptable salt thereof, is a compound named [2(R), 3a(R), 7a(R)]-2,3,3a,4,5,6,7,7a-octahydroindol-2-carboxylic acid, or a pharmaceutically acceptable salt thereof.

95. The method according to Embodiment 81, wherein the 2,3,3a,4,5,6,7,7a-octahydroindol-2-carboxylic acid, or a pharmaceutically acceptable salt thereof, is a compound named [2(R), 3a(S), 7a(S)]-2,3,3a,4,5,6,7,7a-octahydroindol-2-carboxylic acid, or a pharmaceutically acceptable salt thereof.

96. The method according to Embodiment 81, wherein the 2,3,3a,4,5,6,7,7a-octahydroindol-2-carboxylic acid, or a pharmaceutically acceptable salt thereof, is a compound named [2(S), 3a(R), 7a(R)]-2,3,3a,4,5,6,7,7a-octahydroindol-2-carboxylic acid, or a pharmaceutically acceptable salt thereof.

97. The method according to Embodiment 81, wherein the 2,3,3a,4,5,6,7,7a-octahydroindol-2-carboxylic acid, or a pharmaceutically acceptable salt thereof, is a compound named [2(S), 3a(R), 7a(S)]-2,3,3a,4,5,6,7,7a-octahydroindol-2-carboxylic acid, or a pharmaceutically acceptable salt thereof.

98. The method according to Embodiment 81, wherein the 2,3,3a,4,5,6,7,7a-octahydroindol-2-carboxylic acid, or a pharmaceutically acceptable salt thereof, is a compound named [2(R), 3a(S), 7a(R)]-2,3,3a,4,5,6,7,7a-octahydroindol-2-carboxylic acid, or a pharmaceutically acceptable salt thereof.

99. The method according to Embodiment 81, wherein the 2,3,3a,4,5,6,7,7a-octahydroindol-2-carboxylic acid, or a pharmaceutically acceptable salt thereof, is a compound named [2(S), 3a(S), 7a(R)]-2,3,3a,4,5,6,7,7a-octahydroindol-2-carboxylic acid, or a pharmaceutically acceptable salt thereof.

100. The method according to Embodiment 81, wherein the 2,3,3a,4,5,6,7,7a-octahydroindol-2-carboxylic acid, or a pharmaceutically acceptable salt thereof, is a compound named [2(R), 3a(R), 7a(S)]-2,3,3a,4,5,6,7,7a-octahydroindol-2-carboxylic acid, or a pharmaceutically acceptable salt thereof.

Additional embodiments of the invention include:

101. The method according to any one of Embodiments 81-100, wherein the pain is inflammatory pain.

102. The method according to any one of Embodiments 81-100, wherein the pain is osteoarthritic pain.

103. The method according to any one of Embodiments 81 - 100, wherein the pain is caused by cartilage damage.

This invention also provides:

104. A pharmaceutical composition, comprising a 2,3,3a,4,5,6,7,7a-octahydroindol-2-carboxylic acid, or a pharmaceutically acceptable salt thereof, and a pharmaceutically acceptable carrier, diluent, or excipient.

Additional invention composition embodiments include:

105. The pharmaceutical composition according to Embodiment 104, wherein the pharmaceutical composition is a solid dosage form suitable for oral administration.

106. The pharmaceutical composition according to Embodiment 105, wherein the solid dosage form is a tablet form.

107. The pharmaceutical composition according to Embodiment 105, wherein the solid dosage form is a capsule form.

108. The pharmaceutical composition according to Embodiment 104, wherein the pharmaceutical composition is a solid dosage form suitable for mixing with a liquid pharmaceutically acceptable carrier for intravenous administration or administration by injection or oral ingestion.

109. A pharmaceutical composition in solid dosage form, comprising a compound named [2(S), 3a(S), 7a(S)]-2,3,3a,4,5,6,7,7a-octahydroindol-2-carboxylic acid, or a pharmaceutically acceptable salt thereof, and a pharmaceutically acceptable carrier, diluent, or excipient.

110. The pharmaceutical composition according to Embodiment 109, wherein the pharmaceutical composition in solid dosage form is suitable for oral administration.

111. The pharmaceutical composition according to Embodiment 110, wherein the solid dosage form is a tablet form.

112. The pharmaceutical composition according to Embodiment 110, wherein the solid dosage form is a capsule form.

113. The pharmaceutical composition according to Embodiment 109, wherein the pharmaceutical composition in solid dosage form is suitable for mixing with a liquid pharmaceutically acceptable carrier for intravenous administration or administration by injection or oral ingestion.

114. The pharmaceutical composition according to any one of Embodiments 104 to 113, wherein the 2,3,3a,4,5,6,7,7a-octahydroindol-2-carboxylic acid, or a pharmaceutically acceptable salt thereof, is present in unit dosage form.

115. The pharmaceutical composition according to any one of Embodiments 104 to 113, wherein the 2,3,3a,4,5,6,7,7a-octahydroindol-2-carboxylic acid, or a pharmaceutically acceptable salt thereof, is present in unit dosage form in an amount of from 1 milligram to 1000 milligrams.

116. The pharmaceutical composition according to any one of Embodiments 104 to 113, wherein the 2,3,3a,4,5,6,7,7a-octahydroindol-2-carboxylic acid, or a pharmaceutically acceptable salt thereof, is present in unit dosage form in an amount of from 5 milligrams to 500 milligrams.

117. The pharmaceutical composition according to any one of Embodiments 104 to 113, wherein the 2,3,3a,4,5,6,7,7a-octahydroindol-2-carboxylic acid, or a pharmaceutically acceptable salt thereof, is present in unit dosage form in an amount of from 10 milligrams to 500 milligrams.

118. The pharmaceutical composition according to any one of Embodiments 104 to 113, wherein the 2,3,3a,4,5,6,7,7a-octahydroindol-2-carboxylic acid, or a pharmaceutically acceptable salt thereof, is present in unit dosage form in an amount of from 20 milligrams to 500 milligrams.

119. The pharmaceutical composition according to any one of Embodiments 104 to 113, wherein the 2,3,3a,4,5,6,7,7a-octahydroindol-2-carboxylic acid, or a pharmaceutically acceptable salt thereof, is present in unit dosage form in an amount of from 25 milligrams to 250 milligrams.

120. The pharmaceutical composition according to any one of Embodiments 104 to 113, wherein the 2,3,3a,4,5,6,7,7a-octahydroindol-2-carboxylic acid, or a pharmaceutically acceptable salt thereof, is present in unit dosage form in an amount of from 50 milligrams to 250 milligrams.

121. The pharmaceutical composition according to any one of Embodiments 104 to 113, wherein the 2,3,3a,4,5,6,7,7a-octahydroindol-2-carboxylic acid, or a pharmaceutically acceptable salt thereof, is present in unit dosage form in an amount of from 50 milligrams to 200 milligrams.

122. The pharmaceutical composition according to any one of Embodiments 104 to 113, wherein the 2,3,3a,4,5,6,7,7a-octahydroindol-2-carboxylic acid, or a pharmaceutically acceptable salt thereof, is present in unit dosage form in an amount of from 50 milligrams to 100 milligrams.

123. The pharmaceutical composition according to any one of Embodiments 104 to 113, wherein the 2,3,3a,4,5,6,7,7a-octahydroindol-2-carboxylic acid, or a pharmaceutically acceptable salt thereof, is present in unit dosage form in an amount of from 5 milligrams to 50 milligrams.

124. An additional invention embodiment is a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Additional invention embodiments include:

125. A mixture of 2,3,3a,4,5,6,7,7a-octahydroindol-2-carboxylic acids, or pharmaceutically acceptable salts thereof, which is a mixture of any two of the eight possible stereoisomers, or a pharmaceutically acceptable salt thereof.

126. A mixture of 2,3,3a,4,5,6,7,7a-octahydroindol-2-carboxylic acids, or pharmaceutically acceptable salts thereof, which is a mixture of any three of the eight possible stereoisomers, or a pharmaceutically acceptable salt thereof.

127. A mixture of 2,3,3a,4,5,6,7,7a-octahydroindol-2-carboxylic acids, or pharmaceutically acceptable salts thereof, which is a mixture of any four of the eight possible stereoisomers, or a pharmaceutically acceptable salt thereof.

128. A mixture of 2,3,3a,4,5,6,7,7a-octahydroindol-2-carboxylic acids, or pharmaceutically acceptable salts thereof, which is a mixture of any five of the eight possible stereoisomers, or a pharmaceutically acceptable salt thereof.

129. A mixture of 2,3,3a,4,5,6,7,7a-octahydroindol-2-carboxylic acids, or pharmaceutically acceptable salts thereof, which is a mixture of any six of the eight possible stereoisomers, or a pharmaceutically acceptable salt thereof.

130. A mixture of 2,3,3a,4,5,6,7,7a-octahydroindol-2-carboxylic acids, or pharmaceutically acceptable salts thereof, which is a mixture of any seven of the eight possible stereoisomers, or a pharmaceutically acceptable salt thereof.

131. A mixture of 2,3,3a,4,5,6,7,7a-octahydroindol-2-carboxylic acids, or pharmaceutically acceptable salts thereof, which is a mixture of

[2(S), 3a(S), 7a(S)]-2,3,3a,4,5,6,7,7a-octahydroindol-2-carboxylic acid and

[2(R), 3a(R), 7a(R)]-2,3,3a,4,5,6,7,7a-octahydroindol-2-carboxylic acid, or a pharmaceutically acceptable salt thereof.

132. A mixture of 2,3,3a,4,5,6,7,7a-octahydroindol-2-carboxylic acids, or pharmaceutically acceptable salts thereof, which is a mixture of

[2(R), 3a(S), 7a(S)]-2,3,3a,4,5,6,7,7a-octahydroindol-2-carboxylic acid and

[2(S), 3a(R), 7a(R)]-2,3,3a,4,5,6,7,7a-octahydroindol-2-carboxylic acid, or a pharmaceutically acceptable salt thereof.

133. A mixture of 2,3,3a,4,5,6,7,7a-octahydroindol-2-carboxylic acids, or pharmaceutically acceptable salts thereof, which is a mixture of

[2(S), 3a(R), 7a(S)]-2,3,3a,4,5,6,7,7a-octahydroindol-2-carboxylic acid and

[2(R), 3a(S), 7a(R)]-2,3,3a,4,5,6,7,7a-octahydroindol-2-carboxylic acid, or a pharmaceutically acceptable salt thereof.

134. A mixture of 2,3,3a,4,5,6,7,7a-octahydroindol-2-carboxylic acids, or pharmaceutically acceptable salts thereof, which is a mixture of

[2(S), 3a(S), 7a(R)]-2,3,3a,4,5,6,7,7a-octahydroindol-2-carboxylic acid and

[2(R), 3a(R), 7a(S)]-2,3,3a,4,5,6,7,7a-octahydroindol-2-carboxylic acid, or a pharmaceutically acceptable salt thereof.

Additional embodiments of the invention include:

135. A method of treating cartilage damage in a mammal, comprising administering to the mammal a cartilage damage treating effective amount of a 2,3,3a,4,5,6,7,7a-octahydroindol-2-carboxylic acid, or a pharmaceutically acceptable salt thereof, according to any one of Embodiments 124 to 134.

136. A method of preventing cartilage damage in a mammal, comprising administering to the mammal a cartilage damage preventing effective amount of a 2,3,3a,4,5,6,7,7a-octahydroindol-2-carboxylic acid, or a pharmaceutically acceptable salt thereof, according to any one of Embodiments 124 to 134.

137. A method of treating osteoarthritis in a mammal, comprising administering to the mammal an osteoarthritis treating effective amount of a 2,3,3a,4,5,6,7,7a-octahydroindol-2-carboxylic acid, or a pharmaceutically acceptable salt thereof, according to any one of Embodiments 124 to 134.

138. A method of preventing osteoarthritis in a mammal, comprising administering to the mammal an osteoarthritis preventing effective amount of a 2,3,3a,4,5,6,7,7a-octahydroindol-2-carboxylic acid, or a pharmaceutically acceptable salt thereof, according to any one of Embodiments 124 to 134.

139. A method of alleviating pain in a mammal, comprising administering to the mammal a pain alleviating effective amount of a 2,3,3a,4,5,6,7,7a-octahydroindol-2-carboxylic acid, or a pharmaceutically acceptable salt thereof, according to any one of Embodiments 124 to 134.

140. A pharmaceutical composition, comprising a mixture of 2,3,3a,4,5,6,7,7a-octahydroindol-2-carboxylic acids, or a pharmaceutically acceptable salt thereof, according to any one of Embodiments 124 to 134, and a pharmaceutically acceptable carrier, diluent, or excipient.

141. A pharmaceutical composition according to any one of Embodiments 105 to 123, wherein the 2,3,3a,4,5,6,7,7a-octahydroindol-2-carboxylic acid, or a pharmaceutically acceptable salt thereof, comprises a mixture of 2,3,3a,4,5,6,7,7a-octahydroindol-2-carboxylic acids, or a pharmaceutically acceptable salt thereof, according to any one of Embodiments 124 to 134.

Another invention embodiment is:

142. A combination of valdecoxib and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Additional invention embodiments include:

143. A combination of valdecoxib and a 2,3,3a,4,5,6,7,7a-octahydroindol-2-carboxylic acid, or a pharmaceutically acceptable salt thereof, according to any one of Embodiments 124 to 134.

Another invention embodiment is:

144. A method of inhibiting cartilage damage in a mammal, comprising administering to the mammal a therapeutically effective amount of a combination of valdecoxib and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

145. A method of preventing cartilage damage in a mammal, comprising administering to the mammal a therapeutically effective amount of a combination of valdecoxib and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

146. A method of treating osteoarthritis in a mammal, comprising administering to the mammal a therapeutically effective amount of a combination of valdecoxib and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

147. A method of preventing osteoarthritis in a mammal, comprising administering to the mammal a therapeutically effective amount of a combination of valdecoxib and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

148. A method of alleviating pain in a mammal, comprising administering to the mammal a therapeutically effective amount of a combination of valdecoxib and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

149. A pharmaceutical composition, comprising valdecoxib and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof, together with a pharmaceutically acceptable carrier, diluent, or excipient.

Another invention embodiment is:

150. A combination of etanercept and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Additional invention embodiments include:

151. A combination of etanercept and a 2,3,3a,4,5,6,7,7a-octahydroindol-2-carboxylic acid, or a pharmaceutically acceptable salt thereof, according to any one of Embodiments 124 to 134.

Another invention embodiment is:

152. A method of inhibiting cartilage damage in a mammal, comprising administering to the mammal a therapeutically effective amount of a combination of etanercept and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

153. A method of preventing cartilage damage in a mammal, comprising administering to the mammal a therapeutically effective amount of a combination of etanercept and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

154. A method of treating osteoarthritis in a mammal, comprising administering to the mammal a therapeutically effective amount of a combination of etanercept and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

155. A method of preventing osteoarthritis in a mammal, comprising administering to the mammal a therapeutically effective amount of a combination of etanercept and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

156. A method of alleviating pain in a mammal, comprising administering to the mammal a therapeutically effective amount of a combination of etanercept and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

157. A pharmaceutical composition, comprising etanercept and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof, together with a pharmaceutically acceptable carrier, diluent, or excipient.

Another invention embodiment is:

158. A combination of infliximab and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Additional invention embodiments include:

159. A combination of infliximab and a 2,3,3a,4,5,6,7,7a-octahydroindol-2-carboxylic acid, or a pharmaceutically acceptable salt thereof, according to any one of Embodiments 124 to 134.

Another invention embodiment is:

160. A method of inhibiting cartilage damage in a mammal, comprising administering to the mammal a therapeutically effective amount of a combination of infliximab and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

161. A method of preventing cartilage damage in a mammal, comprising administering to the mammal a therapeutically effective amount of a combination of infliximab and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

162. A method of treating osteoarthritis in a mammal, comprising administering to the mammal a therapeutically effective amount of a combination of infliximab and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

163. A method of preventing osteoarthritis in a mammal, comprising administering to the mammal a therapeutically effective amount of a combination of infliximab and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

164. A method of alleviating pain in a mammal, comprising administering to the mammal a therapeutically effective amount of a combination of infliximab and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

165. A pharmaceutical composition, comprising infliximab and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof, together with a pharmaceutically acceptable carrier, diluent, or excipient.

Another invention embodiment is:

166. A combination of methotrexate and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Additional invention embodiments include:

167. A combination of methotrexate and a 2,3,3a,4,5,6,7,7a-octahydroindol-2-carboxylic acid, or a pharmaceutically acceptable salt thereof, according to any one of Embodiments 124 to 134.

Another invention embodiment is:

168. A method of inhibiting cartilage damage in a mammal, comprising administering to the mammal a therapeutically effective amount of a combination of methotrexate and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

169. A method of preventing cartilage damage in a mammal, comprising administering to the mammal a therapeutically effective amount of a combination of methotrexate and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

170. A method of treating osteoarthritis in a mammal, comprising administering to the mammal a therapeutically effective amount of a combination of methotrexate and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

171. A method of preventing osteoarthritis in a mammal, comprising administering to the mammal a therapeutically effective amount of a combination of methotrexate and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

172. A method of alleviating pain in a mammal, comprising administering to the mammal a therapeutically effective amount of a combination of methotrexate and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof.

Another invention embodiment is:

173. A pharmaceutical composition, comprising methotrexate and a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid, or a pharmaceutically acceptable salt thereof, together with a pharmaceutically acceptable carrier, diluent, or excipient.

In a further embodiment, the patient being treated for cartilage damage does not have pain in a joint. In another embodiment, the patient being treated for cartilage damage also has pain in a joint.

In another further embodiment, the patient being treated for inflammation has joint inflammation.

In another further embodiment, the patient being treated for pain has joint pain.

### DETAILED DESCRIPTION OF THE INVENTION

This invention provides a method of preventing or inhibiting cartilage damage in a mammal, comprising administering a cartilage damage inhibiting effective amount of a 2,3,3a,4,5,6,7,7a-octahydroindol-2-carboxylic acid, or a pharmaceutically acceptable salt thereof. This invention also provides a pharmaceutical composition, comprising a 2,3,3a,4,5,6,7,7a-octahydroindol-2-carboxylic acid, or a pharmaceutically acceptable salt thereof, and a pharmaceutically acceptable carrier, diluent, or excipient. This invention also provides a method of preventing or treating osteoarthritis in a mammal, comprising administering a therapeutically effective amount of a 2,3,3a,4,5,6,7,7a-octahydroindol-2-carboxylic acid, or a pharmaceutically acceptable salt thereof. This invention also provides a method of alleviating pain in a mammal, comprising administering a therapeutically effective amount of a 2,3,3a,4,5,6,7,7a-octahydroindol-2-carboxylic acid, or a pharmaceutically acceptable salt thereof. This invention also provides a pharmaceutical composition, comprising a 2,3,3a,4,5,6,7,7a-octahydroindol-2-carboxylic acid, or a pharmaceutically acceptable salt thereof, and a pharmaceutically acceptable carrier, diluent, or excipient. This invention also provides mixtures of 2,3,3a,4,5,6,7,7a-octahydroindol-2-carboxylic acids, or a pharmaceutically acceptable salt thereof. This invention also provides a combination of valdecoxib and a 2,3,3a,4,5,6,7,7a-octahydroindol-2-carboxylic acid, or a pharmaceutically acceptable salt thereof, pharmaceutical compositions containing the said combination, and methods of using the combination. This invention also provides a combination of etanercept and a 2,3,3a,4,5,6,7,7a-octahydroindol-2-carboxylic acid, or a pharmaceutically acceptable salt thereof, pharmaceutical compositions containing the said combination, and methods of using the combination. This invention also provides a combination of infliximab and a 2,3,3a,4,5,6,7,7a-octahydroindol-2-carboxylic acid, or a pharmaceutically acceptable salt thereof, pharmaceutical compositions containing the said combination, and methods of using the combination. This invention also provides a combination of methotrexate and a 2,3,3a,4,5,6,7,7a-octahydroindol-2-carboxylic acid, or a pharmaceutically acceptable salt thereof, pharmaceutical compositions containing the said combination, and methods of using the combination.

The compounds utilized in the method of the present invention are capable of further forming pharmaceutically acceptable salts, including, but not limited to, acid addition and/or base salts. The acid addition salts are formed from basic compounds, whereas the base addition salts are formed from acidic compounds. All of these forms are within the scope of the compounds useful in the method of the present invention.

Pharmaceutically acceptable acid addition salts of a compound useful in the method of the present invention include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are salts of amino acids such as arginate and the like and gluconate, galacturonate (see, for example, Berge S. M. et al., “Pharmaceutical Salts,” J. of Pharma. Sci., 1977;66:1).

An acid addition salt of a compound useful in the method of the present invention is prepared by contacting the free base form of the compound with a sufficient amount of a desired acid to produce a nontoxic salt in the conventional manner. The free base form of the compound may be regenerated by contacting the acid addition salt so formed with a base, and isolating the free base form of the compound in the conventional manner. The free base forms of compounds prepared according to a process of the present invention differ from their respective acid addition salt forms somewhat in certain physical properties such as solubility, crystal structure, hygroscopicity, and the like, but otherwise free base forms of the compounds and their respective acid addition salt forms are equivalent for purposes of the present invention.

A pharmaceutically acceptable base addition salt of a compound useful in the method of the present invention may be prepared by contacting the free acid form of the compound with a nontoxic metal cation such as an alkali or alkaline earth metal cation, or an amine, especially an organic amine. Examples of suitable metal cations include sodium cation (Na⁺), potassium cation (K⁺), magnesium cation (Mg²⁺), calcium cation (Ca²⁺), and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, supra., 1977).

A base addition salt of a compound useful in the method of the present invention may be prepared by contacting the free acid form of the compound with a sufficient amount of a desired base to produce the salt in the conventional manner. The free acid form of the compound may be regenerated by contacting the salt form so formed with an acid, and isolating the free acid of the compound in the conventional manner. The free acid forms of the compounds useful in the method of the present invention differ from their respective salt forms somewhat in certain physical properties such as solubility, crystal structure, hygroscopicity, and the like, but otherwise the salts are equivalent to their respective free acid for purposes of the present invention.

The compounds useful in the method of the present invention can exist in unsolvated forms as well as solvated forms, including hydrated forms. In general, the solvated forms, including hydrated forms, are equivalent to unsolvated forms and are intended to be encompassed within the scope of the present invention.

The compounds useful in the method of the present invention may possess one or more chiral centers, and each center may exist in the R or S configuration. A method of the present invention may utilize any diastereomeric, enantiomeric, or epimeric form of a 2,3,3a,4,5,6,7,7a-octahydroindol-2-carboxylic acid, or a pharmaceutically acceptable salt thereof, as well as mixtures thereof.

The invention method also utilizes isotopically-labelled compounds, which are identical to those recited above, but for the fact that one or more atoms are replaced by an atom having an atomic mass or mass number different from the atomic mass or mass number usually found in nature. Examples of isotopes that can be incorporated into compounds utilized in the invention method include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. Compounds utilized in the present invention method and pharmaceutically acceptable salts of said compounds which contain the aforementioned isotopes and/or other isotopes of other atoms are within the scope of this invention. Certain isotopically labelled compounds utilized in the present invention, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of those described above in this invention can generally be prepared by carrying out the procedures incorporated by reference above and below, or procedures disclosed in the Schemes and/or in the Examples and Preparations, if any, below, by substituting a readily available isotopically labelled reagent for a non-isotopically labelled reagent.

The compounds useful in the method of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib and rofecoxib, analgesics and intraarticular therapies such as corticosteroids and hyaluronic acids such as hyalgan and synvisc.

This invention also relates to a method of or a pharmaceutical composition for treating inflammatory processes and diseases comprising administering a compound useful in the method of this invention to a mammal, including a human, cat, livestock or dog, wherein said inflammatory processes and diseases are defined as above and said inhibitory compound is used in combination with one or more other therapeutically active agents under the following conditions:

A.) where a joint has become seriously inflamed as well as infected at the same time by bacteria, fungi, protozoa and/or virus, said inhibitory combination is administered in combination with one or more antibiotic, antifungal, antiprotozoal and/or antiviral therapeutic agents;

B.) where a multi-fold treatment of pain and inflammation is desired, said inhibitory combination is administered in combination with inhibitors of other mediators of inflammation, comprising one or more members independently selected from the group consisting essentially of:

(1) NSAIDs;

(2) H₁-receptor antagonists;

(3) kinin-B₁- and B₂ -receptor antagonists;

(4) prostaglandin inhibitors selected from the group consisting of PGD-, PGF-PGI₂-and PGE-receptor antagonists;

(5) thromboxane A₂ (TXA₂-) inhibitors;

(6) 5-, 12- and 15-lipoxygenase inhibitors;

(7) leukotriene LTC₄-, LTD₄/LTE₄- and LTB₄ -inhibitors;

(8) PAF-receptor antagonists;

(9) gold in the form of an aurothio group together with one or more hydrophilic groups;

(10) immunosuppressive agents selected from the group consisting of cyclosporine, azathioprine and methotrexate;

(11) anti-inflammatory glucocorticoids;

(12) penicillamine;

(13) hydroxychloroquine;

(14) anti-gout agents including colchicine; xanthine oxidase inhibitors including allopurinol; and uricosuric agents selected from probenecid, sulfinpyrazone and benzbromarone;

C. where older mammals are being treated for disease conditions, syndromes and symptoms found in geriatric mammals, said inhibitory combination is administered in combination with one or more members independently selected from the group consisting essentially of:

(1) cognitive therapeutics to counteract memory loss and impairment;

(2) anti-hypertensives and other cardiovascular drugs intended to offset the consequences of atherosclerosis, hypertension, myocardial ischemia, angina, congestive heart failure and myocardial infarction, selected from the group consisting of:

a. diuretics;

b. vasodilators;

c. β-adrenergic receptor antagonists;

d. angiotensin-II converting enzyme inhibitors (ACE-inhibitors), alone or optionally together with neutral endopeptidase inhibitors;

e. angiotensin II receptor antagonists;

f. renin inhibitors;

g. calcium channel blockers;

h. sympatholytic agents;

i. α₂-adrenergic agonists;

j. α-adrenergic receptor antagonists; and

k. HMG-CoA-reductase inhibitors (anti-hypercholesterolemics);

(3) antineoplastic agents selected from:

a. antimitotic drugs selected from:

i. vinca alkaloids selected from:

[1] vinblastine and

[2] vincristine;

(4) growth hormone secretagogues;

(5) strong analgesics;

(6) local and systemic anesthetics; and

(7) H₂-receptor antagonists, proton pump inhibitors and other gastroprotective agents.

The compounds useful in the method of the present invention may be administered in combination with inhibitors of other mediators of inflammation, comprising one or more members selected from the group consisting essentially of the classes of such inhibitors and examples thereof which include, matrix metalloproteinase inhibitors, aggrecanase inhibitors, TACE inhibitors, leucotriene receptor antagonists, IL-1 processing and release inhibitors, ILra, H₁-receptor antagonists; kinin-B₁- and B₂-receptor antagonists; prostaglandin inhibitors such as PGD-, PGF- PGI₂- and PGE-receptor antagonists; thromboxane A₂ (TXA2-) inhibitors; 5- and 12-lipoxygenase inhibitors; leukotriene LTC₄-, LTD₄/LTE₄ -and LTB₄ -inhibitors; PAF-receptor antagonists; MEK inhibitors; IKK inhibitors; MKK inhibitors; gold in the form of an aurothio group together with various hydrophilic groups; immunosuppressive agents, e.g., cyclosporine, azathioprine and methotrexate; anti-inflammatory glucocorticoids; penicillamine; hydroxychloroquine; anti-gout agents, e.g., colchicine, xanthine oxidase inhibitors, e.g., allopurinol and uricosuric agents, e.g., probenecid, sulfinpyrazone and benzbromarone.

The compounds useful in the method of the present invention may also be used in combination with anticancer agents such as endostatin and angiostatin or cytotoxic drugs such as adriamycin, daunomycin, cis-platinum, etoposide, taxol, taxotere and alkaloids, such as vincristine and antimetabolites such as methotrexate.

The compounds useful in the method of the present invention may also be used in combination with anti-hypertensives and other cardiovascular drugs intended to offset the consequences of atherosclerosis, including hypertension, myocardial ischemia including angina, congestive heart failure and myocardial infarction, selected from vasodilators such as hydralazine, β-adrenergic receptor antagonists such as propranolol, calcium channel blockers such as nifedipine, α₂-adrenergic agonists such as clonidine, α-adrenergic receptor antagonists such as prazosin and HMG-CoA-reductase inhibitors (anti-hypercholesterolemics) such as lovastatin or atorvastatin.

The compounds useful in the method of the present invention may also be administered in combination with one or more antibiotic, antifungal, antiprotozoal, antiviral or similar therapeutic agents.

The compounds useful in the method of the present invention may also be used in combination with CNS agents such as antidepressants (such as sertraline), anti-Parkinsonian drugs (such as L-dopa, requip, mirapex, MAOB inhibitors such as selegine and rasagiline, comP inhibitors such as Tasmar, A-2 inhibitors, dopamine reuptake inhibitors, NMDA antagonists, nicotine agonists, dopamine agonists and inhibitors of neuronal nitric oxide synthase) and anti-Alzheimer's drugs such as donepezil, tacrine, COX-2 inhibitors, propentofylline or metryfonate.

The compounds useful in the method of the present invention may also be used in combination with osteoporosis agents such as roloxifene, lasofoxifene, droloxifene or fosomax and immunosuppressant agents such as FK-506 and metrapamycin.

The present invention also relates to the formulation of the compounds useful in the method of the present invention alone or with one or more other therapeutic agents which are to form the intended combination, including wherein said different drugs have varying half-lives, by creating controlled-release forms of said drugs with different release times which achieves relatively uniform dosing; or, in the case of non-human patients, a medicated feed dosage form in which said drugs used in the combination are present together in admixture in the feed composition. There is further provided in accordance with the present invention co-administration in which the combination of drugs is achieved by the simultaneous administration of said drugs to be given in combination; including co-administration by means of different dosage forms and routes of administration; the use of combinations in accordance with different but regular and continuous dosing schedules whereby desired plasma levels of said drugs involved are maintained in the patient being treated, even though the individual drugs making up said combination are not being administered to said patient simultaneously.

The invention method is useful in human and veterinary medicines for treating osteoarthritis or inhibiting cartilage damage in a mammal, and for treating any other disease or disorder wherein cartilage damage is a symptom or is involved in the underlying pathology of the condition being treated.

The terms and phrases used herein are as defined below or as they otherwise occur in the specification.

As used herein, the phrase “a 2,3,3a,4,5,6,7,7a-octahydroindol-2-carboxylic acid” means a compound selected from:

[2(S), 3a(S), 7a(S)]-2,3,3a,4,5,6,7,7a-octahydroindol-2-carboxylic acid;

[2(R), 3a(R), 7a(R)]-2,3,3a,4,5,6,7,7a-octahydroindol-2-carboxylic acid;

[2(R), 3a(S), 7a(S)]-2,3,3a,4,5,6,7,7a-octahydroindol-2-carboxylic acid;

[2(S), 3a(R), 7a(R)]-2,3,3a,4,5,6,7,7a-octahydroindol-2-carboxylic acid;

[2(S), 3a(R), 7a(S)]-2,3,3a,4,5,6,7,7a-octahydroindol-2-carboxylic acid;

[2(R), 3a(S), 7a(R)]-2,3,3a,4,5,6,7,7a-octahydroindol-2-carboxylic acid;

[2(S), 3a(S), 7a(R)]-2,3,3a,4,5,6,7,7a-octahydroindol-2-carboxylic acid; and

[2(R), 3a(R), 7a(S)]-2,3,3a,4,5,6,7,7a-octahydroindol-2-carboxylic acid.

For illustration purposes, the compound named [2(S), 3a(S), 7a(S)]-2,3,3a,4,5,6,7,7a-octahydroindol-2-carboxylic acid has the structure drawn below:

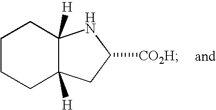

The compound named [2(R), 3a(R), 7a(R)]-2,3,3a,4,5,6,7,7a-octahydroindol-2-carboxylic acid has the structure drawn below:

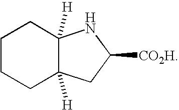

It should be appreciated that the 2,3,3a,4,5,6,7,7a-octahydroindole ring system employs the following numbering scheme:

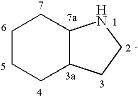

The term “Etanercept” means a dimeric fusion protein consisting of the extracellular ligand-binding portion of the human 75 kilodalton (“p75”) tumor necrosis factor receptor (“TNFR”) linked to the Fc portion of human IgG1. The Fc component of etanercept contains the C_{H}2 domain, the C_{H}3 domain and hinge region, but not the C_{H}1 domain of IgG1. Etanercept is produced by recombinant DNA technology in a Chinese hamster ovary (“CHO”) mammalian cell expression system. It consists of 934 amino acids and has an apparent molecular weight of approximately 150 kilodaltons. Etanercept is an inhibitor of tumor necrosis factor alpha (“TNFalpha”).

Etanercept is marketed in the United States under the tradename ENBREL® for the treatment of rheumatoid arthritis and psoriatic arthritis. ENBREL is supplied as a sterile, white, preservative-free, lyophilized powder for parenteral administration after reconstitution with 1 mL of the supplied Sterile Bacteriostatic Water for Injection, USP (containing 0.9% benzyl alcohol). Following reconstitution, the solution of ENBREL is clear and colorless, with a pH of 7.4±0.3. Each single-use vial of ENBREL contains 25 mg etanercept, 40 mg mannitol, 10 mg sucrose, and 1.2 mg tromethamine.

The term “infliximab” includes the product marketed in the United States under the tradename REMICADE® for the treatment of rheumatoid arthritis.

The term “methotrexate” includes a compound named N-[4-[[(2,4-diamino-6-pteridinyl)methyl]methylamino]benzoyl]-L-glutamic acid, or a pharmaceutically acceptable salt thereof. Methotrexate is used in the treatment of certain neoplastic diseases, severe psoriasis, and adult rheumatoid arthritis. For example, Methotrexate Sodium Tablets for oral administration are available in a packaging system designated as the RHEUMATREX® Methotrexate Sodium Dose Pack for therapy with a weekly dosing schedule of 5 mg, 7.5 mg, 10 mg, 12.5 mg, and 15 mg of methotrexate and the following pharmaceutically acceptable excipients, diluents, or carriers: lactose, magnesium stearate, and pregelatinized starch. The tablets may also contain cornstarch. Methotrexate is also administered by injection intramuscularly, intravenously, intra-arterially, or intrathecally.

The term “valdecoxib” means the compound named 4-(5-methyl-3-phenyl-4-isoxazolyl)-benzenesulfonamide. Valdecoxib has the structure drawn below:

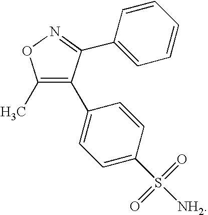

Valdecoxib is a cyclooxygenase-2 (“COX-2”) specific inhibitor that was approved in 2001 by the United States Food and Drug Administration (“FDA”) for treating the signs and symptoms of osteoarthritis and adult rheumatoid arthritis (RA); and the treatment of pain associated with menstrual cramping. Further, valdecoxib is in clinical trials for the treatment of migraine. Valdecoxib tablets are marketed under the tradename BEXTRA®. In a combined analysis of various clinical studies with valdecoxib, valdecoxib was well tolerated with an overall upper gastrointestinal (“GI”) safety profile (ulcers, perforations, obstructions and GI bleeds) significantly better than the conventional NSAIDs studied such as ibuprofen, diclofenac and naproxen.

It should be appreciated that two forms of cyclooxygenase (“COX”) are now known, namely a constitutive isoform usually named cyclooxygenase-1 (“COX-1”) and an inducible isoform usually named cyclooxygenase-2 (“COX-2”), the latter of which expression is upregulated at sites of inflammation. COX-1 appears to play a physiological role and to be responsible for gastrointestinal and renal protection. On the other hand, COX-2 appears to play a pathological role and is believed to be the predominant isoform present in inflammation conditions. The therapeutic use of conventional COX inhibitors, which are typically nonselective inhibitors of both COX-1 and COX-2, is limited due to drug associated side effects, including life threatening ulceration and renal toxicity. Compounds that selectively inhibit COX-2 would exert anti-inflammatory effects without the adverse side effects associated with COX-1 inhibition.

The term “mixture” when used in the context of a description of an invention embodiment refers to two or more 2,3,3a,4,5,6,7,7a-octahydroindol-2-carboxylic acids, or independently selected pharmaceutically acceptable salts thereof, and includes racemic mixtures of enantiomers, mixtures of enantiomers that are not racemic, including mixtures containing from 0.0001% to 99.9999% of one enantiomer and, conversely from 99.9999% to 0.0001% of the other enantiomer, wherein the total amount of 2,3,3a,4,5,6,7,7a-octahydroindol-2-carboxylic acids is 100%, and mixtures of diastercomers, including mixtures containing from 0.0001% to 99.9999% of one diastereomer and, conversely from 99.9999% to 0.0001% of another diastereomer, wherein the total amount of 2,3,3a,4,5,6,7,7a-octahydroindol-2-carboxylic acids is 100%.

It should be appreciated that the terms “uses”, “utilizes”, and “employs”, and their derivatives thereof, are used interchangeably when describing an embodiment of the present invention.

The term “drugs” includes a 2,3,3a,4,5,6,7,7a-octahydroindol-2-carboxylic acid, or a pharmaceutically acceptable salt thereof, and may further include one or two of the other therapeutic agents described above.

The term “ED₄₀” means the dose of a drug, including a 2,3,3a,4,5,6,7,7a-octahydroindol-2-carboxylic acid, or a pharmaceutically acceptable salt thereof, that is sufficient to inhibit cartilage damage or treat a disease or disorder listed above, in at least 40% of the patients being treated.

The term “patient” means a mammal.

For the purposes of this invention, the term “mammal” includes humans, companion animals such as cats and dogs, livestock animals such as horses, cows, pigs, goats, and sheep, and laboratory animals such as guinea pigs, rabbits, rats, mice, hamsters, and monkeys, and transgenic variants thereof.

The phrase “companion animals” includes dogs, cats, rabbits, hamsters, monkeys, horses, and other household or barnyard pets.

The phrase “livestock animals” as used herein refers to domesticated quadrupeds, which includes those being raised for meat and various byproducts, e.g., a bovine animal including cattle and other members of the genus Bos, a porcine animal including domestic swine and other members of the genus Sus, an ovine animal including sheep and other members of the genus Ovis, domestic goats and other members of the genus Capra; domesticated quadrupeds being raised for specialized tasks such as use as a beast of burden, e.g., an equine animal including domestic horses and other members of the family Equidae, genus Equus, or for searching and sentinel duty, e.g., a canine animal including domestic dogs and other members of the genus Canis; and domesticated quadrupeds being raised primarily for recreational purposes, e.g., members of Equus and Canis, as well as a feline animal including domestic cats and other members of the family Felidae, genus Felis.

For the purposes of this invention, the term “arthritis” includes osteoarthritis, rheumatoid arthritis, degenerative joint disease, spondyloarthropathies, gouty arthritis, systemic lupus erythematosus, juvenile arthritis, and psoriatic arthritis.

The phrase “cartilage damage” means a disorder of hyaline cartilage and subchondral bone characterized by hypertrophy of tissues in and around the involved joints, which may or may not be accompanied by deterioration of hyaline cartilage surface. Cartilage damage of OA is thus noninflammatory.

The phrase “inhibiting cartilage damage” means the therapeutic effect of a compound or a combination as defined above that inhibits the progress, prevents further progress, or reverses progression, in part or in whole, of any one or more symptoms of cartilage damage observed for any of the diseases and disorders which have cartilage damage as a component of the disease or disorder pathology.

The phrase “cartilage damage inhibiting effective amount” means an amount of a compound or a combination as defined above sufficient to inhibit the progress, prevent further progress, or reverse progression, in part or in whole, of any one or more symptoms of cartilage damage that is appreciated or suspected or expected in the particular patient being treated.

The phrase “treating” means administration of one or more of the compounds or combinations according to the invention method as defined above that inhibits the progress, prevents further progress, or reverses progression, in part or in whole, of any one or more of the pathological hallmarks or symptoms of any one of the diseases and disorders being treated, including, but not limited to, the symptoms of cartilage damage, pain, and inflammation.

The phrase “treating osteoarthritis” means administration of one or more of the compounds or combinations according to the invention method as defined above that inhibits the progress, prevents further progress, or reverses progression, in part or in whole, of any one or more symptoms of osteoarthritis, including, but not limited to, the symptoms of cartilage damage, pain, and inflammation.

The phrase “preventing” means administration of one or more of the compounds or combinations to an asymptomatic patient, according to the invention method as defined above to inhibit the onset of the condition being prevented, or once onset has occurred, to inhibit the progress, prevent further progress, or reverse progression, in part or in whole, of any one or more pathological hallmarks of any one of the diseases and disorders being prevented.

The phrase “preventing cartilage damage” means administration of one or more of the compounds or combinations to an asymptomatic patient, according to the invention method as defined above to inhibit the onset of the condition being prevented, or once onset has occurred, to inhibit the progress, prevent further progress, or reverse progression, in part or in whole, of any one or more pathological hallmarks of cartilage damage.

The phrase “preventing osteoarthritis” means administration of one or more of the compounds or combinations to an asymptomatic patient, according to the invention method as defined above to inhibit the onset of the condition being prevented, or once onset has occurred, to inhibit the progress, prevent further progress, or reverse progression, in part or in whole, of any one or more pathological hallmarks of osteoarthritis.

The phrase “pain alleviating” means the effect of one or more of the compounds or combinations according to the invention method as defined above that suppresses, reduces, prevents, or otherwise inhibits pain in a patient, including, but not limited to, the suppression, reduction, prevention, or inhibition of pain symptoms due to cartilage damage, inflammatory pain, and pain associated with autoimmune disorders.

It should be appreciated that the invention method can be employed prophylactically to prevent or inhibit the onset of osteoarthritis and cartilage damage in a mammal. Mammals especially in need of prophylactic treatment may be readily identified by one skilled in the medical and pharmaceutical arts by assessing certain risk factors associated with the particular condition being prevented. These risk factors include patient family history of cartilage damage or osteoarthritis, participation in sports or other physically demanding activities such as carpentry, foundry work, and the like, and genetic risk factors.

The phrases “therapeutically effective amount” and “effective amount” are synonymous and mean an amount of a compound or a combination as described above sufficient to inhibit the progress, prevent further progress, or reverse progression, in part or in whole, of any one or more symptoms of the disease or disorder that is appreciated or suspected or expected in the particular patient being treated.

In determining what constitutes a therapeutically effective amount of a 2,3,3a,4,5,6,7,7a-octahydroindol-2-carboxylic acid, or a pharmaceutically acceptable salt thereof, or a combination of the same with valdecoxib, for treating osteoarthritis or inhibiting cartilage damage according to the invention method, a number of factors will generally be considered by the medical practitioner or veterinarian in view of the experience of the medical practitioner or veterinarian, published clinical studies, the subject's (ie, mammal's) age, sex, weight and general condition, as well as the type and extent of the disease, disorder or condition being treated, and the use of other medications, if any, by the subject. Such amounts will generally be from about 0.1 mg/kg to about 300 mg/kg of subject body weight. Typical doses will be from about 10 to about 5000 mg/day for an adult subject of normal weight. In a clinical setting, regulatory agencies such as, for example, the FDA in the United States may require a particular therapeutically effective amount.

As such, the administered dose may fall within the ranges or amounts recited above, or may vary outside, ie, either below or above, those ranges depending upon the requirements of the individual subject, the severity of the condition being treated, and the particular therapeutic formulation being employed. Determination of a proper dose for a particular situation is within the skill of the medical or veterinary arts. Generally, treatment may be initiated using smaller dosages of a 2,3,3a,4,5,6,7,7a-octahydroindol-2-carboxylic acid, or a pharmaceutically acceptable salt thereof, or a combination of the same with valdecoxib, that are less than optimum for a particular subject. Thereafter, the dosage can be increased by small increments until the optimum effect under the circumstance is reached. For convenience, the total daily dosage may be divided and administered in portions during the day, if desired.

The invention method may be conducted by administering a 2,3,3a,4,5,6,7,7a-octahydroindol-2-carboxylic acid, or a pharmaceutically acceptable salt thereof, or a combination of the same with valdecoxib, either alone or formulated in a composition suitable for pharmaceutical administration. Pharmaceutical compositions, described here briefly and more fully below, of a 2,3,3a,4,5,6,7,7a-octahydroindol-2-carboxylic acid, or a pharmaceutically acceptable salt thereof, are produced by formulating the active compound in dosage unit form with a pharmaceutical carrier. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses.

Some examples of suitable pharmaceutical carriers, including pharmaceutical diluents, are gelatin capsules; sugars such as lactose and sucrose; starches such as corn starch and potato starch; cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations.

The compositions to be employed in the invention can also contain other components such as coloring agents, flavoring agents, and/or preservatives. These materials, if present, are usually used in relatively small amounts. The compositions can, if desired, also contain other therapeutic agents commonly employed to treat osteoarthritis. Further, the compositions can, if desired, also contain other therapeutic agents commonly employed to treat secondary symptoms such as, for example, inflammation or pain that may or may not accompany cartilage damage. For example, the compositions may contain aspirin, naproxen, or similar anti-inflammatory analgesic agents.

The percentage of the active ingredients in the foregoing compositions can be varied within wide limits, but for practical purposes it is preferably present in a concentration of at least 10% in a solid composition and at least 2% in a primary liquid composition. The most satisfactory compositions are those in which a much higher proportion of the active ingredient is present, for example, up to about 95%.

Preferred routes of administration of a 2,3,3a,4,5,6,7,7a-octahydroindol-2-carboxylic acid, or a pharmaceutically acceptable salt thereof, or a combination of the same with valdecoxib, are oral or parenteral. For example, a useful intravenous dose is between 5 and 50 mg, and a useful oral dosage is between 20 and 800 mg. The dosage is within the dosing range used in treatment of diseases resulting in cartilage damage such as osteoarthritis, or as would be determined by the physician according to the needs of the patient as described above.

The 2,3,3a,4,5,6,7,7a-octahydroindol-2-carboxylic acid, or a pharmaceutically acceptable salt thereof, or a combination of the same with valdecoxib, may be administered in any form. Preferably, administration is in unit dosage form. A unit dosage form of the a 2,3,3a,4,5,6,7,7a-octahydroindol-2-carboxylic acid, or a pharmaceutically acceptable salt thereof, to be used in this invention may also comprise other compounds useful in the therapy of diseases resulting in cartilage damage.

The advantages of the instant invention include the relatively nontoxic nature of the 2,3,3a,4,5,6,7,7a-octahydroindol-2-carboxylic acids, the ease of preparation, the fact that the compounds are well-tolerated, and the ease of IV and oral administration of the drugs. Further, typically the compounds are not metabolized in the body.

Another important advantage is that the 2,3,3a,4,5,6,7,7a-octahydroindol-2-carboxylic acids provide disease modifying activity for osteoarthritis and other diseases and disorders having cartilage damage as a component of their pathology. There is currently no recognized drug on the market that is being used for this activity.

Further, the instant invention may, if desired, allow the amount of an anti-inflammatory agent and/or pain relieving agent used in the treatment of patients suffering from cartilage damage and inflammation and/or pain to be reduced or even eliminated. It is known that anti-inflammatory and analgesic agents may produce undesirable side effects such as gastro-intestinal bleeding and ulceration. These side effects may be avoided, reduced or eliminated by using the instant invention to inhibit cartilage damage.

Intermediates for the synthesis of a 2,3,3a,4,5,6,7,7a-octahydroindol-2-carboxylic acid, or a pharmaceutically acceptable salt thereof, useful in the invention method, and pharmaceutically acceptable salts thereof, may be prepared by one of ordinary skill in the art of organic chemistry by adapting various synthetic procedures that are well-known in the art of organic chemistry. These synthetic procedures may be found in the literature in, for example, Reagents for Organic Synthesis, by Fieser and Fieser, John Wiley & Sons, Inc, New York, 2000; Comprehensive Organic Transformations, by Richard C. Larock, VCH Publishers, Inc, New York, 1989; the series Compendium of Organic Synthetic Methods, 1989,by Wiley-Interscience; the text Advanced Organic Chemistry, 4^{th} edition, by Jerry March, Wiley-Interscience, New York,1992; or the Handbook of Heterocyclic Chemistry by Alan R. Katritzky, Pergamon Press Ltd, London, 1985, to name a few. Alternatively, a skilled artisan may find methods useful for preparing the intermediates in the chemical literature by searching widely available databases such as, for example, those available from the Chemical Abstracts Service, Columbus, Ohio, or MDL Information Systems GmbH (formerly Beilstein Information Systems GmbH), Frankfurt, Germany.

Preparations of the compounds useful in a method of the present invention may use starting materials, reagents, solvents, and catalysts that may be purchased from commercial sources or they may be readily prepared by adapting procedures in the references or resources cited above. Commercial sources of starting materials, reagents, solvents, and catalysts useful in preparing invention compounds include, for example, The Aldrich Chemical Company, and other subsidiaries of Sigma-Aldrich Corporation, St. Louis, Mo., BACHEM, BACHEM A. G., Switzerland, or Lancaster Synthesis Ltd, United Kingdom.

Syntheses of some compounds useful in the method of the present invention may utilize starting materials, intermediates, or reaction products that contain a reactive functional group. During chemical reactions, a reactive functional group may be protected using protecting groups that render the reactive group substantially inert to the reaction conditions employed. A protecting group is introduced onto a starting material prior to carrying out the reaction step for which a protecting group is needed. Once the protecting group is no longer needed, the protecting group can be removed. It is well within the ordinary skill in the art to introduce protecting groups during a synthesis of a 2,3,3a,4,5,6,7,7a-octahydroindol-2-carboxylic acid, or a pharmaceutically acceptable salt thereof, and then later remove them. Procedures for introducing and removing protecting groups are known and referenced such as, for example, in Protective Groups in Organic Synthesis, 2^{nd} ed., Greene T. W. and Wuts P. G., John Wiley & Sons, New York: New York, 1991, which is hereby incorporated by reference. Thus, for example, protecting groups such as the following may be utilized to protect amino, hydroxyl, and other groups: carboxylic acyl groups such as, for example, formyl, acetyl, and trifluoroacetyl; alkoxycarbonyl groups such as, for example, ethoxycarbonyl, tert-butoxycarbonyl (BOC), β,β,β-trichloroethoxycarbonyl (TCEC), and β-iodoethoxycarbonyl; aralkyloxycarbonyl groups such as, for example, benzyloxycarbonyl (CBZ), para-methoxybenzyloxycarbonyl, and 9-fluorenylmethyloxycarbonyl (FMOC); trialkylsilyl groups such as, for example, trimethylsilyl (TMS) and tert-butyldimethylsilyl (TBDMS); and other groups such as, for example, triphenylmethyl (trityl), tetrahydropyranyl, vinyloxycarbonyl, ortho-nitrophenylsulfenyl, diphenylphosphinyl, para-toluenesulfonyl (Ts), mesyl, trifluoromethanesulfonyl, and benzyl. Examples of procedures for removal of protecting groups include hydrogenolysis of CBZ groups using, for example, hydrogen gas at 50 psi in the presence of a hydrogenation catalyst such as 10% palladium on carbon, acidolysis of BOC groups using, for example, hydrogen chloride in dichloromethane, trifluoroacetic acid (TFA) in dichloromethane, and the like, reaction of silyl groups with fluoride ions, and reductive cleavage of TCEC groups with zinc metal.

Preparations of a 2,3,3a,4,5,6,7,7a-octahydroindol-2-carboxylic acid, or a pharmaceutically acceptable salt thereof, useful in the method of the present invention are incorporated by reference to the patents or patent application publications described above and below.

Certain preparations of the 2,3,3a,4,5,6,7,7a-octahydroindol-2-carboxylic acids are described in U.S. Pat. Nos. 4,691,022; 4,879,392; 4,914,214; 4,935,525; 4,954,640; 5,008,400; 5,101,039; and 5,258,525, which are incorporated by reference herein.

Other preparations of the 2,3,3a,4,5,6,7,7a-octahydroindol-2-carboxylic acids are described in European Patent Numbers 0,037,231; 0,084,164; 0,115,345; 0,173,199; and 0,132,580, which are incorporated by reference herein.

Other preparations of the 2,3,3a,4,5,6,7,7a-octahydroindol-2-carboxylic acids are described in Patent Cooperation Treaty (“PCT”) Application Publication Numbers WO 93/13066, and references cited therein; and WO 00/40555, which are incorporated by reference herein.

Another preparation method is described in the Journal of Medicinal Chemistry, 1987;30:992-998, which is incorporated by reference herein.

The newly discovered ability of a 2,3,3a,4,5,6,7,7a-octahydroindol-2-carboxylic acid, or a pharmaceutically acceptable salt thereof, to inhibit cartilage damage, alleviate pain, and treat osteoarthritis has been established in animal models as described below.

### BIOLOGICAL METHOD 1

Monosodium Iodoacetate-induced Osteoarthritis in Rat Model of Cartilage Damage (“MIA Rat”):

One end result of the induction of osteoarthritis in this model, as determined by histologic analysis, is the development of an osteoarthritic condition within the affected joint, as characterized by the loss of Toluidine blue staining and formation of osteophytes. Associated with the histologic changes is a concentration-dependent degradation of joint cartilage, as evidenced by affects on hind-paw weight distribution of the limb containing the affected joint, the presence of increased amounts of proteoglycan or hydroxyproline in the joint upon biochemical analysis, or histopathological analysis of the osteoarthritic lesions. The compounds 2,3,3a,4,5,6,7,7a-octahydroindol-2-carboxylic acids, or a pharmaceutically acceptable salt thereof, are not effective for relieving pain when administered in an acute model, such as the instant MIA Rat model, which has a duration of just 14 or 28 days, the hind-paw weight distribution effects observed below, or the effects that would be expected to be observed, for the 2,3,3a,4,5,6,7,7a-octahydroindol-2-carboxylic acids, or a pharmaceutically acceptable salt thereof, result from the 2,3,3a,4,5,6,7,7a-octahydroindol-2-carboxylic acids, or a pharmaceutically acceptable salt thereof, ability to directly inhibit damage to cartilage.

Generally, in the MIA Rat model on Day 0, the hind-paw weight differential between the right arthritic joint and the left healthy joint of male Wistar rats (150 g) are determined with an incapacitance tester, model 2KG (Linton Instrumentation, Norfolk, United Kingdom). The incapacitance tester has a chamber on top with an outwardly sloping front wall that supports a rat's front limbs, and two weight sensing pads, one for each hind paw, that facilitates this determination. Then the rats are anesthetized with isofluorine, and the right, hind leg knee joint is injected with 1.0 mg of mono-iodoacetate (“MIA”) through the infrapatellar ligament. Injection of MIA into the joint results in the inhibition of glycolysis and eventual death of surrounding chondrocytes. The rats are further administered either a 2,3,3a,4,5,6,7,7a-octahydroindol-2-carboxylic acid, or a pharmaceutically acceptable salt thereof, or vehicle (in the instant case, water) daily for 14 days or 28 days. The 2,3,3a,4,5,6,7,7a-octahydroindol-2-carboxylic acid, or a pharmaceutically acceptable salt thereof, is typically administered at a dose of 30 mg of 2,3,3a,4,5,6,7,7a-octahydroindol-2-carboxylic acid, or a pharmaceutically acceptable salt thereof, per kilogram of rat per day (30 mg/kg/day), but may be administered at other doses such as, for example, 10 mg/kg/day, 60 mg/kg/day, 90-mg/kg/day, or 100 mg/kg/day according to the requirements of the compound being studied. It is well within the level of ordinary skill in the pharmaceutical arts to determine a proper dosage of a 2,3,3a,4,5,6,7,7a-octahydroindol-2-carboxylic acid, or a pharmaceutically acceptable salt thereof, in this model. Administration of the 2,3,3a,4,5,6,7,7a-octahydroindol-2-carboxylic acid, or a pharmaceutically acceptable salt thereof, in this model is optionally by oral administration or intravenous administration via an osmotic pump. After 7 and 14 days for a two week study, or 7, 14, and 28 days for a four week study, the hind-paw weight distribution is again determined. Typically, the animals administered vehicle alone place greater weight on their unaffected left hind paw than on their right hind paw, while animals administered a 2,3,3a,4,5,6,7,7a-octahydroindol-2-carboxylic acid, or a pharmaceutically acceptable salt thereof, show a more normal (i.e., more like a healthy animal) weight distribution between their hind paws. This change in weight distribution was proportional to the degree of joint cartilage damage. Percent inhibition of a change in hind paw joint function is calculated as the percent change in hind-paw weight distribution for treated animals versus control animals. For example, for a two week study,

$$Percent inhibition of a change in hind paw joint function = \{ 1 - [ \frac{( Δ {W}_{G} )}{( Δ {W}_{C} )} ] \} \times 100$$

wherein: ΔW_{C} is the hind-paw weight differential between the healthy left limb and the arthritic limb of the control animal administered vehicle alone, as measured on Day 14; and

ΔW_{G} is the hind-paw weight differential between the healthy left limb and the arthritic limb of the animal administered a 2,3,3a,4,5,6,7,7a-octahydroindol-2-carboxylic acid, or a pharmaceutically acceptable salt thereof, as measured on Day 14.

In order to measure biochemical or histopathological end points in the MIA Rat model, some of the animals in the above study may be sacrificed, and the amounts of free proteoglycan in both the osteoarthritic right knee joint and the contralateral left knee joint may be determined by biochemical analysis. The amount of free proteoglycan in the contralateral left knee joint provides a baseline value for the amount of free proteoglycan in a healthy joint. The amount of proteoglycan in the osteoarthritic right knee joint in animals administered a 2,3,3a,4,5,6,7,7a-octahydroindol-2-carboxylic acid, and the amount of proteoglycan in the osteoarthritic right knee joint in animals administered vehicle alone, are independently compared to the amount of proteoglycan in the contralateral left knee joint. The amounts of proteoglycan lost in the osteoarthritic right knee joints are expressed as percent loss of proteoglycan compared to the contralateral left knee joint control. The percent inhibition of proteoglycan loss, may be calculated as {[(proteoglycan loss from joint (%) with vehicle)—(proteoglycan loss from joint with 2,3,3a,4,5,6,7,7a-octahydroindol-2-carboxylic acid)]÷(proteoglycan loss from joint (%) with vehicle)}×100. The MIA Rat data that are expected from the analysis of proteoglycan loss would establish that 2,3,3a,4,5,6,7,7a-octahydroindol-2-carboxylic acids, including [2(S), 3a(S), 7a(S)]-2,3,3a,4,5,6,7,7a-octahydroindol-2-carboxylic, are effective for inhibiting cartilage damage and treating osteoarthritis in mammalian patients, including human.

### BIOLOGICAL METHOD 2

[2(S), 3a(S), 7a(S)]-2,3,3a,4,5,6,7,7a-octahydroindol-2-carboxylic in MIA:

In a particular experiment, monosodium iodoacetate (“MIA”) (1 mg/joint) was injected through the infrapatellar ligament of the right knee of anesthetized male, Wistar rats. The contralateral control knee was injected with 50 μL of physiologic saline. The change in hind paw weight distribution, as determined by use of an incapacitance tester, between the right (arthritic) and left (contralateral control) knees was utilized as an index of functional limitation in the arthritic knee. Limitations in joint function were determined on days 7, 14, and 28 following induction of arthritis. Following sacrifice, erosion severity was determined on the tibial plateaus from the arthritic joint. Histological analysis was also conducted on these samples. The basis of the invention is derived from the ability of [2(S), 3a(S), 7a(S)]-2,3,3a,4,5,6,7,7a-octahydroindol-2-carboxylic acid, dosed orally two times per day (i.e., PO; BID), to significantly decrease cartilage erosion severity at 30-mg/kg and 10-mg/kg doses and by its ability to decrease joint function limitations as defined by a reduction in differential hind-limb weight bearing.

For oral administration, [2(S), 3a(S), 7a(S)]-2,3,3a,4,5,6,7,7a-octahydroindol-2-carboxylic acid was dissolved in double distilled water (all calculations are based on the percent parent of the drug). Dose-response studies ranging from 3 to 30 mg/kg (PO; BID) demonstrated that [2(S), 3a(S), 7a(S)]-2,3,3a,4,5,6,7,7a-octahydroindol-2-carboxylic acid, at 4 weeks post-MIA, significantly decreased the degree of structural damage to the cartilage at the 30 mg/kg and 10 mg/kg doses and significantly decreased joint pain at all doses.

The results of these studies with oral dosing are shown below in Table 1 in the columns labelled “IJFL (%+/−SEM)”, wherein IJFL means Inhibition of Joint Function Limitation, “SDCES”, wherein SDCES means Significant Decrease In Cartilage Erosion Severity, and “SIJWHLE”, wherein SIJWHLE means Significant Increase in Joints Without Hind Limb Erosion.

TABLE 1
Four week study with oral administration of [2(S), 3a(S), 7a(S)]-
2,3,3a,4,5,6,7,7a-octahydroindol-2-carboxylic acid two times per
day per dose:

| Dose | IJFL | | |
|---|---|---|---|
| (mg/kg) | (% +/− SEM)^{a} | SDCES^{b} | SIJWHLE^{c} |
| 30 | 83 +/− 7 | Yes^{d} | Yes^{e} |
| 10 | 70 +/− 9 | No | Yes^{f} |
| 3 | 62 +/− 6 | No | No |

^{a}p < 0.05 versus vehicle (Student's t-test);
^{b}p < 0.05 versus vehicle (Ridit Analysis);
^{c}p < 0.05 versus vehicle (Exact Sequential Cochran-Armitage Trend test);
^{d}actual p = 0.021;
^{e}actual p = 0.020;
^{f}actual p = 0.046.

The compound [2(S), 3a(S), 7a(S)]-2,3,3a,4,5,6,7,7a-octahydroindol-2-carboxylic acid was also administered subcutaneously via osmotic pumps. Dosing was carried out at 100-mg/kg/day, 90-mg/kg/day, 30-mg/kg/day, and 10-mg/kg/day dosing. The results of these studies with dosing by osmotic pump are shown below in Table 2 in the columns labelled “IJFL (%+/−SEM)”, wherein IJFL means Inhibition of Joint Function Limitation, “SDCES”, wherein SDCES means Significant Decrease In Cartilage Erosion Severity, and “SIJWHLE”, wherein SIJWHLE means Significant Increase in Joints Without Hind Limb Erosion.

TABLE 2
Two and Four week studies with administration of [2(S), 3a(S), 7a(S)]-
2,3,3a,4,5,6,7,7a-octahydroindol-2-carboxylic acid via osmotic pumps:

| Duration | Dose | IJFL | | |
|---|---|---|---|---|
| of study | (mg/kg/day) | (% +/− SEM)^{a} | SDCES^{b} | SIJWHLE^{c} |
| 2 weeks | 90 | 85 +/− 3 | ND^{d} | ND |
| 4 weeks | 100 | 78 +/− 3 | No | No |
| 4 weeks | 30 | 94 +/− 10 | No | No |
| 4 weeks | 10 | 28 +/− 14^{e} | No | No |

^{a}p < 0.05 versus vehicle (Student's t-test);
^{b}p < 0.05 versus vehicle (Ridit Analysis);
^{c}p < 0.05 versus vehicle (Exact Sequential Cochran-Armitage Trend test);
^{d}ND means not determined;
^{e}Not statistically significant.

The proportion of subjects without hind limb erosions was analyzed via an Exact Sequential Cochran-Armitage Trend test (SAS® Institute, 1999). The Cochran-Armitage Trend test is employed when one wishes to determine whether the proportion of positive or “Yes” responders increases or decreases with increasing levels of treatment. For the particular study, it was found that the number of animals without joint erosions increased with increasing dose.

The ridit analysis was used to determine differences in overall erosion severity. This parameter takes into account both the erosion grade (0=no erosion, I=erosion extending into the superficial or middle layers, or II=deep layer erosion), and area (small, medium and large, quantified by dividing the area of the largest erosion in each score into thirds) simultaneously. The analysis recognizes that each unit of severity is different, but does not assume a mathematical relationship between units.

The MIA Rat data reported above in Tables 1 and 2 establish that 2,3,3a,4,5,6,7,7a-octahydroindol-2-carboxylic acids, including [2(S), 3a(S), 7a(S)]-2,3,3a,4,5,6,7,7a-octahydroindol-2-carboxylic, are effective at preventing or treating cartilage damage.

### BIOLOGICAL METHOD 3

Induction of Experimental Osteoarthritis in Rabbit (“EOA in Rabbit”):

Normal rabbits are anaesthetized and anteromedial incisions of the right knees performed. The anterior cruciate ligaments are visualized and sectioned. The wounds are closed and the animals are housed in individual cages, exercised, and fed ad libitum. Rabbits are given either vehicle (water) or a [2(S), 3a(S), 7a(S)]-2,3,3a,4,5,6,7,7a-octahydroindol-2-carboxylic acid, or a pharmaceutically acceptable salt thereof, (10 rabbits per group). Each group was dosed three times per day with the [2(S), 3a(S), 7a(S)]-2,3,3a,4,5,6,7,7a-octahydroindol-2-carboxylic acid, or a pharmaceutically acceptable salt thereof, group receiving 30-mg/kg/dose or 10-mg/kg/dose. The rabbits are euthanized 8 weeks after surgery and the proximal end of the tibia and the distal end of the femur are removed from each animal.

Macroscopic Grading

The cartilage changes on the femoral condyles and tibial plateaus are graded separately under a dissecting microscope (Stereozoom, Bausch & Lomb, Rochester, N.Y.). The depth of erosion is graded on a scale of 0 to 4 as follows: grade 0=normal surface; Grade 1=minimal fibrillation or a slight yellowish discoloration of the surface; Grade 2=erosion extending into superficial or middle layers only; Grade 3=erosion extending into deep layers; Grade 4=erosion extending to subchondral bone. The surface area changes are measured and expressed in mm². Representative specimens may also be used for histologic grading (see below).

Histologic Grading

Histologic evaluation is performed on sagittal sections of cartilage from the lesional areas of the femoral condyle and tibial plateau. Serial sections (5 um) are prepared and stained with safranin-O. The severity of OA lesions is graded on a scale of 0-14 by two independent observers using the histologic-histochemical scale of Mankin et al. This scale evaluates the severity of OA lesions based on the loss of safranin-O staining (scale 0-4), cellular changes (scale 0-3), invasion of tidemark by blood vessels (scale 0-1) and structural changes (scale 0-6). On this latter scale, 0 indicates normal cartilage structure and 6 indicates erosion of the cartilage down to the subchondral bone. The scoring system is based on the most severe histologic changes in the multiple sections.

Representative specimens of synovial membrane from the medial and lateral knee compartments are dissected from underlying tissues. The specimens are fixed, embedded, and sectioned (5 um) as above, and stained with hematoxylin-eosin. For each compartment, two synovial membrane specimens are examined for scoring purposes and the highest score from each compartment is retained. The average score is calculated and considered as a unit for the whole 30 knee. The severity of synovitis is graded on a scale of 0 to 10 by two independent observers, adding the scores of 3 histologic criteria: synovial lining cell hyperplasia (scale 0-2); villous hyperplasia (scale 0-3); and degree of cellular infiltration by mononuclear and polymorphonuclear cells (scale 0-5): 0 indicates normal structure.

Statistical Analysis

Mean values and SEM is calculated and statistical analysis was done using the Mann-Whitney U-test.

The results of these studies would be expected to show that a 2,3,3a,4,5,6,7,7a-octahydroindol-2-carboxylic acid, or a pharmaceutically acceptable salt thereof, including [2(S), 3a(S), 7a(S)]-2,3,3a,4,5,6,7,7a-octahydroindol-2-carboxylic acid, would reduce the size of the lesion on the tibial plateaus, and perhaps the damage in the tibia or on the femoral condyles. In conclusion, these results would show that a 2,3,3a,4,5,6,7,7a-octahydroindol-2-carboxylic acid, or a pharmaceutically acceptable salt thereof, including [2(S), 3a(S), 7a(S)]-2,3,3a,4,5,6,7,7a-octahydroindol-2-carboxylic acid, would have significant inhibition effects on the damage to cartilage.

The foregoing study would establish that a 2,3,3a,4,5,6,7,7a-octahydroindol-2-carboxylic acid, or a pharmaceutically acceptable salt thereof, including [2(S), 3a(S), 7a(S)]-2,3,3a,4,5,6,7,7a-octahydroindol-2-carboxylic acid, are effective for the inhibition of cartilage damage and treatment of osteoarthritis in human, and other mammalian disorders. Such a treatment offers a distinct advantage over existing treatments that only modify pain and other secondary symptoms. The effectiveness of a 2,3,3a,4,5,6,7,7a-octahydroindol-2-carboxylic acid, or a pharmaceutically acceptable salt thereof, including [2(S), 3a(S), 7a(S)]-2,3,3a,4,5,6,7,7a-octahydroindol-2-carboxylic acid, in this model would indicate that a 2,3,3a,4,5,6,7,7a-octahydroindol-2-carboxylic acid, or a pharmaceutically acceptable salt thereof, including [2(S), 3a(S), 7a(S)]-2,3,3a,4,5,6,7,7a-octahydroindol-2-carboxylic acid, will have clinically useful effects in preventing and/or treating cartilage damage.

Administration according to the invention method of a 2,3,3a,4,5,6,7,7a-octahydroindol-2-carboxylic acid, or a pharmaceutically acceptable salt thereof, to a mammal to treat the diseases listed above is preferably, although not necessarily, accomplished by administering the compound, or a salt thereof, in a pharmaceutical dosage form.

The 2,3,3a,4,5,6,7,7a-octahydroindol-2-carboxylic acids, or a pharmaceutically acceptable salt thereof, can be prepared and administered according to the invention method in a wide variety of oral and parenteral pharmaceutical dosage forms. Thus, the 2,3,3a,4,5,6,7,7a-octahydroindol-2-carboxylic acids, or a pharmaceutically acceptable salt thereof, can be administered by injection, that is, intravenously, intramuscularly, intracutaneously, subcutaneously, intraduodenally, or intraperitoneally. Also, the 2,3,3a,4,5,6,7,7a-octahydroindol-2-carboxylic acids, or a pharmaceutically acceptable salt thereof, can be administered by inhalation, for example, intranasally. Additionally, the 2,3,3a,4,5,6,7,7a-octahydroindol-2-carboxylic acids, or a pharmaceutically acceptable salt thereof, can be administered transdermally. It will be obvious to those skilled in the art that the following dosage forms may comprise as the active components either a 2,3,3a,4,5,6,7,7a-octahydroindol-2-carboxylic acid, or a pharmaceutically acceptable salt thereof. The active compounds generally are present in a concentration of about 5% to about 95% by weight of the formulation.

For preparing pharmaceutical compositions from the 2,3,3a,4,5,6,7,7a-octahydroindol-2-carboxylic acids, or a pharmaceutically acceptable salt thereof, (i.e., the active components) pharmaceutically acceptable carriers can be either solid or liquid. Solid form preparations are preferred. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component. Powders suitable for intravenous administration or administration by injection may be lyophilized.

In tablets, the active component is mixed with the carrier having the necessary binding properties in suitable proportions and compacted in the shape and size desired.

The powders and tablets preferably contain from about 5% to about 70%, total, of the active component. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active component with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into unit doses containing an appropriate quantity of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.01 to 1000 mg, preferably 1 to 500 mg according to the particular application and the potency of the active components. The composition can, if desired, also contain other compatible therapeutic agents.

In therapeutic use as agents to treat the above-listed diseases, the 2,3,3a,4,5,6,7,7a-octahydroindol-2-carboxylic acids, or a pharmaceutically acceptable salt thereof, or a combination of the same with valdecoxib, are administered at a dose that is effective for treating at least one symptom of the disease or disorder being treated. The initial dosage of about 1 mg/kg to about 100 mg/kg daily of the active component will be effective. A daily dose range of about 25 mg/kg to about 75 mg/kg of the active component is preferred. The dosages, however, may be varied depending upon the requirements of the patient, the severity of the condition being treated, and the 2,3,3a,4,5,6,7,7a-octahydroindol-2-carboxylic acid, or a pharmaceutically acceptable salt thereof, or combination being employed. Determination of the proper dosage for a particular situation is within the skill of the art as described above. Typical dosages will be from about 0.1 mg/kg to about 500 mg/kg, and ideally about 25 mg/kg to about 250 mg/kg, such that it will be an amount that is effective to treat the particular disease or disorder being treated.

A preferred composition for dogs comprises an ingestible liquid peroral dosage form selected from the group consisting of a solution, suspension, emulsion, inverse emulsion, elixir, extract, tincture and concentrate, optionally to be added to the drinking water of the dog being treated. Any of these liquid dosage forms, when formulated in accordance with methods well known in the art, can either be administered directly to the dog being treated, or may be added to the drinking water of the dog being treated. The concentrate liquid form, on the other hand, is formulated to be added first to a given amount of water, from which an aliquot amount may be withdrawn for administration directly to the dog or addition to the drinking water of the dog.

A preferred composition provides delayed-, sustained- and/or controlled-release of the 2,3,3a,4,5,6,7,7a-octahydroindol-2-carboxylic acid, or a pharmaceutically acceptable salt thereof. Such preferred compositions include all such dosage forms which produce≧40% inhibition of cartilage degradation, and result in a plasma concentration of the active component of at least 3 fold the active component's ED₄₀ for at least 2 hours; preferably for at least 4 hours; preferably for at least 8 hours; more preferably for at least 12 hours; more preferably still for at least 16 hours; even more preferably still for at least 20 hours; and most preferably for at least 24 hours. Preferably, there is included within the above-described dosage forms those which produce≧40% inhibition of cartilage degradation, and result in a plasma concentration of the active component of at least 5 fold the active component's ED₄₀ for at least 2 hours, preferably for at least 2 hours, preferably for at least 8 hours, more preferably for at least 12 hours, still more preferably for at least 20 hours and most preferably for at least 24 hours. More preferably, there is included the above-described dosage forms which produce≧50% inhibition of cartilage degradation, and result in a plasma concentration of the active component of at least 5 fold the active component's ED₄₀ for at least 2 hours, preferably for at least 4 hours, preferably for at least 8 hours, more preferably for at least 12 hours, still more preferably for at least 20 hours and most preferably for at least 24 hours.

The following examples illustrate the invention pharmaceutical compositions containing a cartilage damage treating effective amount or an anti-osteoarthritic effective amount of a 2,3,3a,4,5,6,7,7a-octahydroindol-2-carboxylic acid, and a pharmaceutically acceptable carrier, diluent, or excipient. The examples are representative only, and are not to be construed as limiting the invention in any respect.

### FORMULATION EXAMPLE 1

Tablet Formulation:

| Ingredient | Amount (mg) |
|---|---|
| [2(S), 3a(S), 7a(S)]-2,3,3a,4,5,6,7,7a-octahydroindol-2- | 25 |
| carboxylic acid | |
| Lactose | 50 |
| Cornstarch (for mix) | 10 |
| Cornstarch (paste) | 10 |
| Magnesium stearate (1%) | 5 |
| Total | 100 |

[2(S), 3a(S), 7a(S)]-2,3,3a,4,5,6,7,7a-octahydroindol-2-carboxylic acid, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for inhibiting cartilage damage or treating osteoarthritis.

### FORMULATION EXAMPLE 2

Coated Tablets:

The tablets of Formulation Example 1 are coated in a customary manner with a coating of sucrose, potato starch, talc, tragacanth, and colorant.

### FORMULATION EXAMPLE 3

Injection vials:

The pH of a solution of 500 g of [2(S), 3a(S), 7a(S)]-2,3,3a,4,5,6,7,7a-octahydroindol-2-carboxylic acid and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 25 mg of [2(S), 3a(S), 7a(S)]-2,3,3a,4,5,6,7,7a-octahydroindol-2-carboxylic acid.

### FORMULATION EXAMPLE 4

Suppositories:

A mixture of 25 g of [2(R), 3a(R), 7a(R)]-2,3,3a,4,5,6,7,7a-octahydroindol-2-carboxylic acid, 100 g of soya lecithin, and 1400 g of cocoa butter is fused, poured into molds, and allowed to cool. Each suppository contains 25 mg of[2(R), 3a(R), 7a(R)]-2,3,3a,4,5,6,7,7a-octahydroindol-2-carboxylic acid.

### FORMULATION EXAMPLE 5

Solution:

A solution is prepared from 1 g of [2(S), 3a(R), 7a(R)]-2,3,3a,4,5,6,7,7a-octahydroindol-2-carboxylic acid, 9.38 g of NaH₂PO₄·12H₂O, 28.48 g of Na₂HPO₄·12H₂O, and 0.1 g benzalkonium chloride in 940 mL of double-distilled water. The pH of the solution is adjusted to pH 6.8 using 2 M hydrochloric acid. The solution is diluted to 1.0 L with double-distilled water, and sterilized by irradiation. A 25 mL volume of the solution contains 25 mg of [2(S), 3a(R), 7a(R)]-2,3,3a,4,5,6,7,7a-octahydroindol-2-carboxylic acid.

### FORMULATION EXAMPLE 6

Ointment:

500 mg of [2(R), 3a(S), 7a(R)]-2,3,3a,4,5,6,7,7a-octahydroindol-2-carboxylic acid is mixed with 99.5 g of petroleum jelly under aseptic conditions. A 5 g portion of the ointment contains 25 mg of [2(R), 3a(S), 7a(R)]-2,3,3a,4,5,6,7,7a-octahydroindol-2-carboxylic acid.

### FORMULATION EXAMPLE 7

Capsules:

2 kg of [2(S), 3a(S), 7a(S)]-2,3,3a,4,5,6,7,7a-octahydroindol-2-carboxylic acid are filled into hard gelatin capsules in a customary manner such that each capsule contains 25 mg of [2(S), 3a(S), 7a(S)]-2,3,3a,4,5,6,7,7a-octahydroindol-2-carboxylic acid.

### FORMULATION EXAMPLE 8

Ampoules:

A solution of 2.5 kg of [2(S), 3a(S), 7a(R)]-2,3,3a,4,5,6,7,7a-octahydroindol-2-carboxylic acid is dissolved in 60 L of double-distilled water. The solution is sterile filtered, and the filtrate is filled into ampoules. The ampoules are lyophilized under sterile conditions and aseptically sealed. Each ampoule contains 25 mg of [2(S), 3a(S), 7a(R)]-2,3,3a,4,5,6,7,7a-octahydroindol-2-carboxylic acid.

The following examples illustrate the invention pharmaceutical compositions containing an invention combination and a pharmaceutically acceptable carrier, diluent, or excipient. The examples are representative only, and are not to be construed as limiting the invention in any respect.

### FORMULATION EXAMPLE 9

Tablet Formulation:

| Ingredient | Amount (mg) |
|---|---|
| [2(S), 3a(S), 7a(S)]-2,3,3a,4,5,6,7,7a-octahydroindol-2- | 25 |
| carboxylic acid | |
| Valdecoxib | 20 |
| Lactose | 50 |
| Cornstarch (for mix) | 10 |
| Cornstarch (paste) | 10 |
| Magnesium stearate (1%) | 5 |
| Total | 120 |

[2(S), 3a(S), 7a(S)]-2,3,3a,4,5,6,7,7a-octahydroindol-2-carboxylic acid, valdecoxib, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of one of the above-listed diseases.

### FORMULATION EXAMPLE 10

Coated Tablets:

The tablets of Formulation Example 9 are coated in a customary manner with a coating of sucrose, potato starch, talc, tragacanth, and colorant.

### FORMULATION EXAMPLE 11

Injection vials:

The pH of a solution of 250 g of valdecoxib, 500 g of [2(S), 3a(S), 7a(S)]-2,3,3a,4,5,6,7,7a-octahydroindol-2-carboxylic acid, and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 12.5 mg of valdecoxib and 25 mg of [2(S), 3a(S), 7a(S)]-2,3,3a,4,5,6,7,7a-octahydroindol-2-carboxylic acid.

### FORMULATION EXAMPLE 12

Suppositories:

A mixture of 50 g of valdecoxib, 25 g of [2(R), 3a(R), 7a(R)]-2,3,3a,4,5,6,7,7a-octahydroindol-2-carboxylic acid, 100 g of soya lecithin, and 1400 g of cocoa butter is fused, poured into molds, and allowed to cool. Each suppository contains 50 mg of valdecoxib and 25 mg of [2(R), 3a(R), 7a(R)]-2,3,3a,4,5 ,6,7,7a-octahydroindol-2-carboxylic acid.

### FORMULATION EXAMPLE 13

Solution:

A solution is prepared from 0.5 g of valdecoxib, 1 g of [2(S), 3a(R), 7a(S)]-2,3,3a,4,5,6,7,7a-octahydroindol-2-carboxylic acid, 9.38 g of NaH₂PO₄·12H₂O, 28.48 g of Na₂HPO₄·12H₂O, and 0.1 g benzalkonium chloride in 940 mL of double-distilled water. The pH of the solution is adjusted to pH 6.8 using 2 M hydrochloric acid. The solution is diluted to 1.0 L with double-distilled water, and sterilized by irradiation. A 25 mL volume of the solution contains 12.5 mg of valdecoxib and 25 mg of [2(S), 3a(R), 7a(S)]-2,3,3a,4,5 ,6,7,7a-octahydroindol-2-carboxylic acid.

### FORMULATION EXAMPLE 14

Ointment:

100 mg of valdecoxib, 500 mg of [2(S), 3a(S), 7a(S)]-2,3,3a,4,5,6,7,7a-octahydroindol-2-carboxylic acid is mixed with 99.4 g of petroleum jelly under aseptic conditions. A 5 g portion of the ointment contains 5 mg of valdecoxib and 25 mg of [2(S), 3a(S), 7a(S)]-2,3,3a,4,5,6,7,7a-octahydroindol-2-carboxylic acid.

### FORMULATION EXAMPLE 15

Capsules:

2 kg of valdecoxib and 20 kg of [2(S), 3a(S), 7a(S)]-2,3,3a,4,5,6,7,7a-octahydroindol-2-carboxylic acid are filled into hard gelatin capsules in a customary manner such that each capsule contains 25 mg of valdecoxib and 250 mg of [2(S), 3a(S), 7a(S)]-2,3,3a,4,5,6,7,7a-octahydroindol-2-carboxylic acid.

### FORMULATION EXAMPLE 16

Ampoules:

A solution of 2.5 kg of valdecoxib and 2.5 kg of [2(S), 3a(S), 7a(S)]-2,3,3a,4,5,6,7,7a-octahydroindol-2-carboxylic acid is dissolved in 60 L of double-distilled water. The solution is sterile filtered, and the filtrate is filled into ampoules. The ampoules are lyophilized under sterile conditions and aseptically sealed. Each ampoule contains 25 mg each of valdecoxib and [2(S), 3a(S), 7a(S)]-2,3,3a,4,5,6,7,7a-octahydroindol-2-carboxylic acid.

While it may be desirable to formulate valdecoxib and a [2(S), 3a(S), 7a(S)]-2,3,3a,4,5,6,7,7a-octahydroindol-2-carboxylic acid, or a pharmaceutically acceptable salt thereof, together in one capsule, tablet, ampoule, solution, and the like, for simultaneous administration, it is not necessary for the purposes of practicing the invention methods. Valdecoxib and a [2(S), 3a(S), 7a(S)]-2,3,3a,4,5,6,7,7a-octahydroindol-2-carboxylic acid, or a pharmaceutically acceptable salt thereof, of an invention combination alternatively can each be formulated independently in any form such as, those of any one Formulation Examples 1 to 16, and administered either simultaneously or at different times.

The following examples illustrate the invention pharmaceutical compositions containing discrete formulations of the active components of the invention combination and a pharmaceutically acceptable carrier, diluent, or excipient. The examples are representative only, and are not to be construed as limiting the invention in any respect.

### FORMULATION EXAMPLE 17

Tablet Formulation of [2(S), 3a(S), 7a(S)]-2,3,3a,4,5,6,7,7a-
octahydroindol-2-carboxylic acid:

| Ingredient | Amount (mg) |
|---|---|
| [2(S), 3a(S), 7a(S)]-2,3,3a,4,5,6,7,7a-octahydroindol-2- | 25 |
| carboxylic acid | |
| Lactose | 50 |
| Cornstarch (for mix) | 10 |
| Cornstarch (paste) | 10 |
| Magnesium stearate (1%) | 5 |
| Total | 100 |

[2(S), 3a(S), 7a(S)]-2,3,3a,4,5,6,7,7a-octahydroindol-2-carboxylic acid, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet.

Injection vial formulation of valdecoxib:

The pH of a solution of 500 g of valdecoxib and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 25 mg of valdecoxib.

Such tablets containing [2(S), 3a(S), 7a(S)]-2,3,3a,4,5,6,7,7a-octahydroindol-2-carboxylic acid can be administered to a human from one to four times a day for treatment of the above-listed diseases, and the injection solutions containing valdecoxib can be administered to a human 1 or 2 times per day, wherein the administration by injection is optionally simultaneous with administration of the tablets or at different times, for the treatment of one of the above-listed diseases.

### FORMULATION EXAMPLE 18

Coated Tablets containing [2(S), 3a(S), 7a(S)]-2,3,3a,4,5,6,7,7a-octahydroindol-2-carboxylic acid:

The tablets of Formulation Example 17 are coated in a customary manner with a coating of sucrose, potato starch, talc, tragacanth, and colorant.

Capsules containing valdecoxib:

2 kg of valdecoxib are filled into hard gelatin capsules in a customary manner such that each capsule contains 25 mg of valdecoxib.

Such coated tablets containing [2(S), 3a(S), 7a(S)]-2,3,3a,4,5,6,7,7a-octahydroindol-2-carboxylic acid can be administered to a human from one to four times a day for treatment of the above-listed diseases, and the capsules containing valdecoxib can be administered to a human 1 or 2 times per day, wherein the administration of the capsules is optionally simultaneous with administration of the tablets or at different times, for the treatment of one of the above-listed diseases.

Still further, it should be appreciated that the invention methods comprising administering an invention combination to a mammal to treat diseases or disorders listed above may be used to treat different diseases simultaneously. For example, administration of valdecoxib in accordance with the invention combination may be carried out as described above to treat inflammation, arthritic pain, pain associated with menstrual cramping, and migraines, while a 2,3,3a,4,5,6,7,7a-octahydroindol-2-carboxylic acid, or a pharmaceutically acceptable salt thereof, may be administered to treat OA or inhibit cartilage damage.

As shown above, the invention method offers a distinct advantage over existing treatments for diseases such as OA that comprise cartilage damage, wherein the existing treatments modify pain or secondary symptoms, but do not show a disease modifying effect. The effectiveness of 2,3,3a,4,5,6,7,7a-octahydroindol-2-carboxylic acids, including [2(S), 3a(S), 7a(S)]-2,3,3a,4,5,6,7,7a-octahydroindol-2-carboxylic acid, have been shown to be useful for inhibiting cartilage damage and treating osteoarthritis.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

Having described the invention method, various embodiments of the invention are hereupon claimed.

What is claimed is:

1. A method of inhibiting cartilage damage in a mammal, comprising administering to the mammal a cartilage damage inhibiting effective amount of a 2,3,3a,4,5,6,7,7a-octahydroindol-2-carboxylic acid, or a pharmaceutically acceptable salt thereof.

2. The method according to claim 1, wherein the 2,3,3a,4,5,6,7,7a-octahydroindol-2-carboxylic acid, or a pharmaceutically acceptable salt thereof, is a compound named [2(S), 3a(S), 7a(S)]-2,3,3a,4,5,6,7,7a-octahydroindol-2-carboxylic acid, or a pharmaceutically acceptable salt thereof.

3. A method of treating osteoarthritis in a mammal, comprising administering to the mammal a therapeutically effective amount of a 2,3,3a,4,5,6,7,7a-octahydroindol-2-carboxylic acid, or a pharmaceutically acceptable salt thereof.

4. The method according to claim 3, wherein the 2,3,3a,4,5,6,7,7a-octahydroindol-2-carboxylic acid, or a pharmaceutically acceptable salt thereof, is a compound named [2(S), 3a(S), 7a(S)]-2,3,3a,4,5,6,7,7a-octahydroindol-2-carboxylic acid, or a pharmaceutically acceptable salt thereof.

5. A method of alleviating pain, inflammatory pain, osteoarthritic pain, or pain caused by cartilage damage in a mammal, comprising administering to the mammal a therapeutically effective amount of a 2,3,3a,4,5,6,7,7a-octahydroindol-2-carboxylic acid, or a pharmaceutically acceptable salt thereof.

6. The method according to claim 5, wherein the 2,3,3a,4,5,6,7,7a-octahydroindol-2-carboxylic acid, or a pharmaceutically acceptable salt thereof, is a compound named [2(S), 3a(S), 7a(S)]-2,3,3a,4,5,6,7,7a-octahydroindol-2-carboxylic acid, or a pharmaceutically acceptable salt thereof.

7. A pharmaceutical composition, comprising a 2,3,3a,4,5,6,7,7a-octahydroindol-2-carboxylic acid, or a pharmaceutically acceptable salt thereof, and a pharmaceutically acceptable carrier, diluent, or excipient, wherein the pharmaceutical composition is a solid dosage form suitable for oral administration.

8. The pharmaceutical composition according to claim 7, wherein the 2,3,3a,4,5,6,7,7a-octahydroindol-2-carboxylic acid, or a pharmaceutically acceptable salt thereof, is a compound named [2(S), 3a(S), 7a(S)]-2,3,3a,4,5,6,7,7a-octahydroindol-2-carboxylic acid, or a pharmaceutically acceptable salt thereof.
